(19) United States
(12) Patent Application Publication
(10) Pub. No.: US 2014/0120036 A1
(43) Pub. Date: May 1, 2014

(54) RADIOLABELED 5-HT6 LIGANDS

(71) Applicant: AbbVie Inc., North Chicago, IL (US)

(72) Inventor: Lawrence A. Black, Libertyville, IL (US)

(21) Appl. No.: 13/723,422

(22) Filed: Dec. 21, 2012

Related U.S. Application Data
(60) Provisional application No. 61/583,688, filed on Jan. 6, 2012.

(51) Int. Cl. A61K 51/04; C07D 295/112

(52) U.S. Cl. CPC A61K 51/0459; C07D 295/112
USPC 424/1.89; 544/395; 424/1.81; 424/9.1

(57) ABSTRACT

Compounds of formula (I) are disclosed

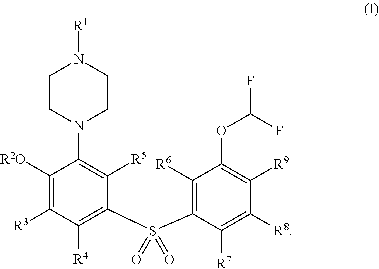

Compounds of formula (I) are useful in treating conditions and disorders prevented by or ameliorated by 5-HT₆ receptor ligands. Radiolabeled compounds of formula (I) are also useful as diagnostic tools as 5-HT₆ positron emission tomography ligands. Also disclosed are pharmaceutical compositions comprising compounds of formula (I), methods for using such compounds and compositions, and a process for preparing compounds within the scope of formula (I).

### CROSS-REFERENCE TO RELATED APPLICATION

This claims priority to U.S. Provisional Patent Application No. 61/583,688, filed on Jan. 6, 2012, the contents of which are herein fully incorporated by reference.

### TECHNICAL FIELD

The invention relates to phenylsulfonyl derivatives, and more particularly, to radiolabeled phenylsulfonyl derivatives, compositions comprising such compounds, methods of using such compounds and compositions, and processes for preparing such compounds.

### BACKGROUND

Serotonin (5-hydroxytryptamine, 5-HT), a monoamine neurotransmitter and local hormone, is formed by the hydroxylation and decarboxylation of tryptophan. The greatest concentration is found in the enterochromaffin cells of the gastrointestinal tract, the remainder being predominantly present in platelets and in the Central Nervous System (CNS). 5-HT is implicated in a vast array of physiological and pathophysiological pathways. In the periphery, it contracts a number of smooth muscles and induces endothelium-dependent vasodilation. In the CNS, it is believed to be involved in a wide range of functions, including the control of appetite, mood, anxiety, hallucinations, sleep, vomiting and pain perception.

Neurons that secrete 5-HT are termed serotonergic. The function of 5-HT is exerted upon its interaction with specific (serotonergic) neurons. Until now, seven types of 5-HT receptors have been identified: 5-HT₁ (with subtypes 5-HT_{1A}, 5-HT_{1B}, 5-HT_{1D}, 5-HT_{1E} and 5-HT_{1F}), 5-HT₂ (with subtypes 5-HT_{2A}, 5-HT_{2B} and 5-HT_{2c}), 5-HT₃, 5-HT₄, 5-HT₅ (with subtypes 5-HT_{5A} and 5-HT_{5B}), 5-HT₆ and 5-HT₇. Most of these receptors are coupled to G-proteins that affect the activities of either adenylate cyclase or phospholipase Cy.

The human 5-HT₆ receptors are positively coupled to adenylyl cyclase. They are distributed throughout the limbic, striatal and cortical regions of the brain and show a high affinity to antipsychotics.

The modulation of the 5-HT₆ receptor by suitable substances is expected to improve certain disorders including cognitive dysfunctions, such as a deficit in memory, cognition and learning, in particular associated with Alzheimer's disease, age-related cognitive decline and mild cognitive impairment, attention deficit disorder/hyperactivity syndrome, personality disorders, such as schizophrenia, in particular cognitive deficits related with schizophrenia, affective disorders such as depression, anxiety and obsessive compulsive disorders, motion or motor disorders such as Parkinson's disease and epilepsy, migraine, sleep disorders (including disturbances of the Circadian rhythm), feeding disorders, such as anorexia and bulimia, certain gastrointestinal disorders such as Irritable Bowl Syndrome, diseases associated with neurodegeneration, such as stroke, spinal or head trauma and head injuries, such as hydrocephalus, drug addiction and obesity.

Although various classes of compounds having an affinity for the 5-HT₆ receptor are known, it would be beneficial to provide additional compounds demonstrating high affinity and selectivity for the 5-HT₆ receptor. The compounds should have low affinity to adrenergic receptors, such as α₁-adrenergic receptor, histamine receptors, such as H₁-receptor, and dopaminergic receptors, such as D₂-receptor, in order to avoid or reduce considerable side effects associated with modulation of these receptors, such as postural hypotension, reflex tachycardia, potentiation of the antihypertensive effect of prazosin, terazosin, doxazosin and labetalol or dizziness associated to the blockade of the α₁-adrenergic receptor, weight gain, sedation, drowsiness or potentiation of central depressant drugs associated to the blockade of the H₁-receptor, or extrapyramidal movement disorder, such as dystonia, parkinsonism, akathisia, tardive dyskinesia or rabbit syndrome, or endocrine effects, such as prolactin elevation (galactorrhea, gynecomastia, menstruyl changes, sexual dysfunction in males), associated to the blockade of the D₂-receptor.

A useful tool for assessing the ability of a compound to modulate a particular receptor in humans and animals is positron emission tomography (PET). Positron emission tomography includes the use of positron or gamma emitting radiolabeled compounds to study the interaction between an unlabeled compound and the radiolabeled compound for binding to the receptor of interest. This information is valuable for clinical candidate selection, determination of first-in-human dosing levels, proof of concept studies, and assessment of probability of success of a drug candidate relative to its therapeutic index. The topic and use of positron-emitting ligands for this purpose has been generally reviewed, for example in “PET ligands for assessing receptor occupancy in vivo” Burns, et al Annual Reports in Medicinal Chemistry (2001), 36, 267-276; “Ligand-receptor interactions as studied by PET: implications for drug development” by Jarmo Hietala, Annals of Medicine (Helsinki) (1999), 31(6), 438-443; “Positron emission tomography neuroreceptor imaging as a tool in drug discovery, research and development” Burns, et al. Current Opinion in Chemical Biology (1999), 3(4), 388-394.

The only validated 5-HT₆ receptor positron emission tomography ligand for clinical use is [¹¹C]GSK-215083, disclosed in WO2006/053785 A1 and EP1824830 B1. However, one disadvantage to using [¹¹C]GSK-215083 as a 5-HT₆ receptor PET ligand is related to the radioligand's lack of selectivity relative to the 5-HT_{2A} receptor. Specifically, [¹¹C]GSK-215083 has a 5-HT₆ Kᵢ of 0.339 nM and a 5-HT₂ₐ Kᵢ of 0.395 nM. In practice, this lack of selectivity requires pretreatment with ketanserin, a selective 5-HT_{2A} antagonist, so that only 5-HT₆ receptors will be imaged by the PET ligand.

Accordingly, it would be beneficial to provide additional compounds useful for noninvasive imaging of 5-HT₆ receptor occupancy in humans and animals. In particular, it would be beneficial to provide 5-HT₆ receptor PET ligands having high affinity and selectivity for 5-HT₆ receptors.

### SUMMARY

This invention is directed to phenylsulfonyl derivatives, and more particularly, to radiolabeled phenylsulfonyl derivatives, compositions comprising such compounds, methods of using such compounds and compositions, and processes for preparing such compounds.

In one aspect, the invention relates to phenylsulfonyl derivatives having a compound of formula (I):

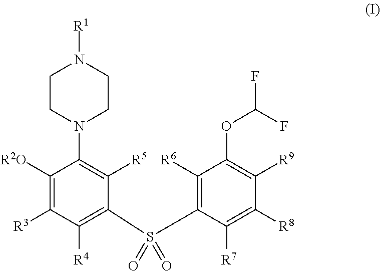

or a pharmaceutically acceptable salt, ester, amide, prodrug, or radiolabeled form thereof, wherein
R¹ and R² are each independently selected from the group consisting of hydrogen, C₁-C₆ alkyl, C₁-C₆ alkenyl, and C₃-C₈ cycloalkyl; and
R³, R⁴, R⁵, R⁶, R⁷, R⁸, and R⁹ are each independently selected from the group consisting of hydrogen, halogen, C₁-C₆ alkyl, C₁-C₆ alkenyl, and C₃-C₈ cycloalkyl.

Another aspect of the invention relates to pharmaceutical compositions comprising compounds of the invention. Such compositions can be administered in accordance with a method of the invention as part of a therapeutic regimen for treatment or prevention of conditions and disorders related to 5-HT₆ receptor activity. Such compositions can be administered in a diagnostic procedure, such as positron emission tomography (PET) or single photon emission computed tomography (sPECT).

Another aspect of the invention relates to use of the compounds and compositions of the invention as diagnostic tools. The compounds of the invention, synthesized with ¹¹C, ¹⁸F, or other positron-emitting isotopes are suitable ligand tools for PET. Especially suitable compounds of the invention for this use are those wherein a ¹¹CH₃ group can be incorporated into the compound by reaction with ¹¹CH₃I or ¹¹CH₃OTf. Also, especially suitable compounds of the use are those wherein a ¹⁸F group can be incorporated into the compound by reaction with ¹⁸F-fluoride anion. The incorporation of ¹¹CH₃ can be carried out according to a method known to those skilled in the art. According to one method, compounds of formula (I) wherein at least one of R¹ and R² is hydrogen, can be treated with base and an alkyl iodide such as ¹¹CH₃I to prepare ligands for use in PET studies. For incorporation of ¹⁸F into compounds or compositions of the invention, compounds of formula (I) wherein at least one of R¹ and R² is hydroxyalkyl such as hydroxyethyl, can be treated with methanesulfonic anhydride or triflic anhydride and a base in an inert solvent such as dichloromethane, and the resulting compound (a methanesulfonate or triflate) can be treated with ¹⁸F-fluoride by methods well known to skilled in the art of synthetic organic chemistry or medicinal chemistry.

Yet another aspect of the invention relates to a method of selectively modulating 5-HT₆ receptor activity. The method is useful for treating, or preventing conditions and disorders related to 5-HT₆ receptor modulation in mammals. More particularly, the method is useful for treating or preventing conditions and disorders related to central nervous system function, including memory, cognition processes and neurological processes.

Processes for making compounds of the invention also are contemplated.

The compounds, compositions comprising the compounds, methods for making the compounds, methods for treating or preventing conditions and disorders by administering the compounds, radiolabeled forms of the compounds, compositions containing radiolabeled forms of the compounds, and methods of using radiolabeled forms of the compounds are further described herein.

### DETAILED DESCRIPTION

### Definition of Terms

Certain terms as used in the specification are intended to refer to the following definitions, as detailed below.

The term “acyl” as used herein means an alkyl group, as defined herein, appended to the parent molecular moiety through a carbonyl group, as defined herein. Representative examples of acyl include, but are not limited to, acetyl, 1-oxopropyl, 2,2-dimethyl-1-oxopropyl, 1-oxobutyl, and 1-oxopentyl.

The term “acyloxy” as used herein means an acyl group, as defined herein, appended to the parent molecular moiety through an oxygen atom. Representative examples of acyloxy include, but are not limited to, acetyloxy, propionyloxy, and isobutyryloxy.

The term “alkenyl” as used herein means a straight or branched chain hydrocarbon containing from 2 to 10 carbons, and preferably 2, 3, 4, 5, or 6 carbons, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxyimino” as used herein means an alkoxy group, as defined herein, appended to the parent molecular moiety through an imino group, as defined herein. Representative examples of alkoxyimino include, but are not limited to, ethoxy(imino)methyl and methoxy(imino)methyl.

The term “alkoxysulfonyl” as used herein means an alkoxy group, as defined herein, appended to the parent molecular moiety through a sulfonyl group, as defined herein. Representative examples of alkoxysulfonyl include, but are not limited to, methoxysulfonyl, ethoxysulfonyl, and propoxysulfonyl.

The term “alkyl” as used herein means a straight or branched chain hydrocarbon containing from 1 to 10 carbon atoms, and preferably 1, 2, 3, 4, 5, or 6 carbons. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. Each of the carbon atoms of the alkyl group is substituted with hydrogen or with 0, 1, or 2 substituents selected from acyl, acyloxy, alkoxy, alkoxyalkoxy, alkoxyalkyl, alkoxycarbonyl, alkoxyimino, alkoxysulfonyl, alkylcarbonyl, alkylsulfonyl, amido, carboxy, cyano, cycloalkyl, fluoroalkoxy, formyl, haloalkoxy, haloalkyl, halogen, hydroxy, hydroxyalkyl, mercapto, nitro, oxo, alkylthio, NR_{A}R_{B}, (NR_{A}R_{B})carbonyl, (NR_{A}R_{B})sulfonyl, —OS(O)₂-alkyl, and —OS(O)₂-aryl.

The term “alkylene” means a divalent group derived from a straight or branched chain hydrocarbon of from 1 to 10 carbon atoms. Representative examples of alkylene include, but are not limited to, —CH₂—, —CH(CH)—, —C(CH₃)₂—, —CH₂CH₂—, —CH₂CH₂CH₂—, —CH₂CH₂CH₂CH₂—, and —CH₂CH(CH)CH₂—.

The term “alkylamino” as used herein means an alkyl group, as defined herein, appended to the parent molecular moiety through a NH group. Representative examples of alkylamino include, but are not limited to, methylamino, ethylamino, isopropylamino, and butylamino.

The term “alkylcarbonyl” as used herein means an alkyl group, as defined herein, appended to the parent molecular moiety through a carbonyl group, as defined herein. Representative examples of alkylcarbonyl include, but are not limited to, methylcarbonyl, ethylcarbonyl, isopropylcarbonyl, n-propylcarbonyl, and the like.

The term “alkylsulfonyl” as used herein means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein means a straight or branched chain hydrocarbon group containing from 2 to 10 carbon atoms, and preferably 2, 3, 4, or 5 carbons, and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “amido” as used herein means an amino, alkylamino, or dialkylamino group appended to the parent molecular moiety through a carbonyl group, as defined herein. Representative examples of amido include, but are not limited to, aminocarbonyl, methylaminocarbonyl, dimethylaminocarbonyl, and ethylmethylaminocarbonyl.

The term “amino” as used herein means an —NH₂ group.

The term “aryl,” as used herein, means phenyl, a bicyclic aryl, or a tricyclic aryl. The bicyclic aryl is attached to the parent molecular moiety through any carbon atom contained within the bicyclic aryl. Representative examples of the bicyclic aryl include, but are not limited to, dihydroindenyl, indenyl, naphthyl, dihydronaphthalenyl, and tetrahydronaphthalenyl. The tricyclic aryl is a tricyclic aryl ring system such as anthracene or phenanthrene, a bicyclic aryl fused to a cycloalkyl, a bicyclic aryl fused to a cycloalkenyl, or a bicyclic aryl fused to a phenyl. The tricyclic aryl is attached to the parent molecular moiety through any carbon atom contained within the tricyclic aryl. Representative examples of tricyclic aryl ring include, but are not limited to, anthracenyl, phenanthrenyl, azulenyl, dihydroanthracenyl, fluorenyl, and tetrahydrophenanthrenyl.

The carbon atoms of the aryl groups of this invention are substituted with hydrogen or are optionally substituted with one or more substituents independently selected from acyl, acyloxy, alkenyl, alkoxy, alkoxyalkoxy, alkoxyalkyl, alkoxycarbonyl, alkoxyimino, alkoxysulfonyl, alkyl, alkylcarbonyl, alkylsulfonyl, alkynyl, amido, carboxy, cyano, cycloalkyl, fluoroalkoxy, formyl, haloalkoxy, haloalkyl, halogen, hydroxy, hydroxyalkyl, mercapto, nitro, alkylthio, —NR_{A}R_{B}, (NR_{A}R_{B})carbonyl, —SO₂N(R₁₄ₐ)(R_{14b}), and N(R₁₄)SO₂(R_{14b}). Where the aryl group is a phenyl group, the number of substituents is 0, 1, 2, 3, 4, or 5. Where the aryl group is a bicyclic aryl, the number of substituents is 0, 1, 2, 3, 4, 5, 6, 7, 8, or 9. Where the aryl group is a tricyclic aryl, the number of substituents is 0, 1, 2, 3, 4, 5, 6, 7, 8, or 9.

The term “arylalkyl” as used herein means an aryl group, as defined herein, appended to the parent molecular moiety through an alkyl group, as defined herein. Representative examples of arylalkyl include, but are not limited to, benzyl, 2-phenylethyl and 3-phenylpropyl.

The term “carbonyl” as used herein means a —C(═O)— group.

The term “carboxy” as used herein means a —CO₂H group, which may be protected as an ester group —CO₂-alkyl.

The term “cyano” as used herein means a —CN group, attached to the parent molecular moiety through the carbon.

The term “cyanophenyl” as used herein means a —CN group appended to the parent molecular moiety through a phenyl group, including, but not limited to, 4-cyanophenyl, 3-cyanophenyl, and 2-cyanophenyl.

The term “cycloalkyl” as used herein means a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

Each of the carbon atoms of the cycloalkyl groups of the invention is substituted with 0, 1, or 2 substituents selected from acyl, acyloxy, alkenyl, alkoxy, alkoxyalkoxy, alkoxyalkyl, alkoxycarbonyl, alkoxyimino, alkoxysulfonyl, alkyl, alkylcarbonyl, alkylsulfonyl, alkynyl, amido, carboxy, cyano, cycloalkyl, fluoroalkoxy, formyl, haloalkoxy, haloalkyl, halogen, hydroxy, hydroxyalkyl, mercapto, nitro, alkylthio, —NR_{A}R_{B}, (NR_{A}R_{B})carbonyl, SO₂N(R₁₄ₐ) (R_{14b}), and N(R₁₄ₐ)SO₂(R_{14b}).

The term “cycloalkylcarbonyl” as used herein means a cycloalkyl group, as defined herein, appended to the parent molecular moiety through a carbonyl group, as defined herein. Representative examples of cycloalkylcarbonyl include, but are not limited to, cyclopropylcarbonyl, cyclopentylcarbonyl, cyclohexylcarbonyl, and cycloheptylcarbonyl.

The term “dialkylamino” as used herein means two independent alkyl groups, as defined herein, appended to the parent molecular moiety through a nitrogen atom. Representative examples of dialkylamino include, but are not limited to, dimethylamino, diethylamino, ethylmethylamino, and butylmethylamino.

The term “fluoro” as used herein means —F.

The term “fluoroalkyl” as used herein means at least one fluoro group, as defined herein, appended to the parent molecular moiety through an alkyl group, as defined herein. Representative examples of fluoroalkyl include, but are not limited to, fluoromethyl, difluoromethyl, trifluoromethyl, pentafluoroethyl, 2-fluoroethyl, and 2,2,2-trifluoroethyl.

The term “fluoroalkoxy” as used herein means at least one fluoro group, as defined herein, appended to the parent molecular moiety through an alkoxy group, as defined herein. Representative examples of fluoroalkoxy include, but are not limited to, fluoromethoxy, difluoromethoxy, trifluoromethoxy, pentafluoroethoxy, heptafluoropropyloxy, and 2,2,2-trifluoroethoxy.

The term “formyl” as used herein means a —C(O)H group.

The term “halo” or “halogen” as used herein means Cl, Br, I, or F.

The term “haloalkoxy” as used herein means at least one halogen, as defined herein, appended to the parent molecular moiety through an alkoxy, as defined herein. Representative examples of haloalkoxy include, but are not limited to, 2-fluoroethoxy, trifluoromethoxy, and pentafluoroethoxy.

The term “haloalkyl” as used herein means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle”, as used herein, refers to aromatic or non-aromatic cyclic groups that contain at least one heteroatom. Examples of aromatic heterocycles are, for example, heteroaryl groups as further defined below. Non-aromatic heterocycles are non-aromatic cyclic groups that contain at least one heteroatom; examples of non-aromatic heterocyclic groups or non-aromatic heterocycles are further defined below. Heterocyclic rings are connected to the parent molecular moiety through a carbon atom, or alternatively in the case of heterocyclic rings that contain a bivalent nitrogen atom having a free site for attachment, the heterocyclic ring may be connected to the parent molecular moiety though a nitrogen atom. Additionally, the heterocycles may be present as tautomers.

The term “heteroaryl”, as used herein, refers to an aromatic ring containing one or more heteroatoms independently selected from nitrogen, oxygen, or sulfur, or a tautomer thereof. Such rings can be monocyclic or bicyclic as further described herein.

The terms “monocyclic heteroaryl” or “5- or 6-membered heteroaryl ring”, as used herein, refer to 5- or 6-membered aromatic rings containing 1, 2, 3, or 4 heteroatoms independently selected from nitrogen, oxygen, or sulfur, or a tautomer thereof. Examples of such rings include, but are not limited to, a ring wherein one carbon is replaced with an O or S atom; one, two, or three N atoms are arranged in a suitable manner to provide an aromatic ring; or a ring wherein two carbon atoms in the ring are replaced with one O or S atom and one N atom. Such rings can include, but are not limited to, a six-membered aromatic ring wherein one to four of the ring carbon atoms are replaced by nitrogen atoms, five-membered rings containing a sulfur, oxygen, or nitrogen in the ring; five-membered rings containing one to four nitrogen atoms; and five-membered rings containing an oxygen or sulfur and one to three nitrogen atoms. Representative examples of 5- to 6-membered heteroaryl rings include, but are not limited to, furyl, imidazolyl, isoxazolyl, isothiazolyl, oxazolyl, pyrazinyl, pyrazolyl, pyridazinyl, pyridinyl, pyrimidinyl, pyrrolyl, tetrazolyl, thiadiazolyl, thiadiazolonyl, thiadiazinonyl, oxadiazolyl, oxadiazolonyl, oxadiazinonyl, thiazolyl, thienyl, triazinyl, triazolyl, triazolyl, pyridazinonyl, pyridonyl, and pyrimidinonyl.

The term “bicyclic heteroaryl” or “8- to 12-membered bicyclic heteroaryl ring”, as used herein, refers to an 8-, 9-, 10-, 11-, or 12-membered bicyclic aromatic ring containing at least 3 double bonds, and wherein the atoms of the ring include one or more heteroatoms independently selected from oxygen, sulfur, and nitrogen. Representative examples of bicyclic heteroaryl rings include indolyl, benzothienyl, benzofuranyl, indazolyl, benzimidazolyl, benzothiazolyl, benzoxazolyl, benzoisothiazolyl, benzoisoxazolyl, quinolinyl, isoquinolinyl, quinazolinyl, quinoxalinyl, phthalazinyl, pteridinyl, purinyl, naphthyridinyl, cinnolinyl, thieno[2,3-d]imidazole, 1,5-dihydro-benzo[b][1,4]diazepin-2-on-yl, and pyrrolopyrimidinyl.

Heteroaryl groups of the invention, whether monocyclic or bicyclic, may be substituted with hydrogen, or optionally substituted with one or more substituents independently selected from acyl, acyloxy, alkenyl, alkoxy, alkoxyalkoxy, alkoxyalkyl, alkoxycarbonyl, alkoxyimino, alkoxysulfonyl, alkyl, alkylcarbonyl, alkylsulfonyl, alkynyl, amido, carboxy, cyano, cycloalkyl, fluoroalkoxy, formyl, haloalkoxy, haloalkyl, halogen, hydroxy, hydroxyalkyl, mercapto, nitro, oxo, alkylthio, —NR_{A}R_{B}, (NR_{A}R_{B})carbonyl, —SO₂N(R₁₄ₐ)(R_{14b}), and N(R₁₄ₐ)SO₂(R_{14b}). Monocyclic heteroaryl or 5- or 6-membered heteroaryl rings are substituted with 0, 1, 2, 3, 4, or 5 substituents. Bicyclic heteroaryl or 8- to 12-membered bicyclic heteroaryl rings are substituted with 0, 1, 2, 3, 4, 5, 6, 7, 8, or 9 substituents. Heteroaryl groups of the present invention may be present as tautomers.

The terms “non-aromatic heterocyclic ring” and “non-aromatic heterocycle”, as used herein, refer to a 4- to 12-membered monocyclic or bicyclic ring containing at least one saturated carbon atom, and also containing one, two, three, four, or five heteroatoms independently selected from the group consisting of nitrogen, oxygen, and sulfur. Four- and five-membered rings may have zero or one double bond. Six-membered rings may have zero, one, or two double bonds. Seven- and eight-membered rings may have zero, one, two, or three double bonds. The non-aromatic heterocycle groups of the invention can be attached through a carbon atom or a nitrogen atom. The non-aromatic heterocycle groups may be present in tautomeric form. Representative examples of nitrogen-containing heterocycles include, but are not limited to, azepanyl, azetidinyl, aziridinyl, azocanyl, morpholinyl, piperazinyl, piperidinyl, pyrrolidinyl, pyrrolinyl, dihydrothiazolyl, dihydropyridinyl, and thiomorpholinyl. Representative examples of non-nitrogen containing non-aromatic heterocycles include, but are not limited to, dioxanyl, dithianyl, tetrahydrofuryl, dihydropyranyl, tetrahydropyranyl, and [1,3]dioxolanyl.

The non-aromatic heterocycles of the invention may be substituted with hydrogen, or optionally substituted with 0, 1, 2, 3, 4, 5, 6, 7, 8, or 9 substituents independently selected from acyl, acyloxy, alkenyl, alkoxy, alkoxyalkoxy, alkoxyalkyl, alkoxycarbonyl, alkoxyimino, alkoxysulfonyl, alkyl, alkylcarbonyl, alkylsulfonyl, alkynyl, amido, carboxy, cyano, cycloalkyl, fluoroalkoxy, formyl, haloalkoxy, haloalkyl, halogen, hydroxy, hydroxyalkyl, mercapto, nitro, oxo, alkylthio, —NR_{A}R_{B}, (NR_{A}R_{B})carbonyl, —SO₂N(R₁₄ₐ) (R_{14b}), and N(R₁₄ₐ)SO₂(R_{14b}).

Additional examples of heterocycles include, but are not limited to, isoindoline-1,3-dione, (Z)-1H-benzo[e][1,4]diazepin-5(4H)-one, pyrimidine-2,4(1H,3H)-dione, benzo[d]thiazol-2(3H)-one, pyridin-4(1H)-one, imidazolidin-2-one, 1H-imidazol-2(3H)-one, pyridazin-3(2H)-one, tetrahydropyrimidin-2(1H)-one, and 1H-benzo[d]imidazol-2(3H)-one.

The term “heterocyclealkyl” as used herein means a heterocycle group, as defined herein, appended to the parent molecular moiety through an alkyl group, as defined herein. Representative examples of heterocyclealkyl include, but are not limited to, 2-thienylmethyl, 2-thienylethyl, 2-furylethyl, and 2-furylmethyl.

The term “hydroxy” as used herein means an —OH group.

The term “hydroxyalkyl” as used herein means at least one hydroxy group, as defined herein, appended to the parent molecular moiety through an alkyl group, as defined herein. Representative examples of hydroxyalkyl include, but are not limited to, hydroxymethyl, 2-hydroxyethyl, 2-methyl-2-hydroxyethyl, 3-hydroxypropyl, 2,3-dihydroxypentyl, and 2-ethyl-4-hydroxyheptyl.

The term “hydroxy-protecting group” means a substituent which protects hydroxyl groups against undesirable reactions during synthetic procedures. Examples of hydroxy-protecting groups include, but are not limited to, methoxymethyl, benzyloxymethyl, 2-methoxyethoxymethyl, 2-(trimethylsilyl)ethoxymethyl, benzyl, triphenylmethyl, 2,2,2-trichloroethyl, t-butyl, trimethylsilyl, t-butyldimethylsilyl, t-butyldiphenylsilyl, methylene acetal, acetonide benzylidene acetal, cyclic ortho esters, methoxymethylene, cyclic carbonates, and cyclic boronates. Hydroxy-protecting groups are appended onto hydroxy groups by reaction of the compound that contains the hydroxy group with a base, such as triethylamine, and a reagent selected from an alkyl halide, alkyl trifilate, trialkylsilyl halide, trialkylsilyl triflate, aryldialkylsilyltriflate, or an alkylchloroformate, CH₂I₂, or a dihaloboronate ester, for example with methyliodide, benzyl iodide, triethylsilyltriflate, acetyl chloride, benzylchloride, or dimethylcarbonate. A protecting group also may be appended onto a hydroxy group by reaction of the compound that contains the hydroxy group with acid and an alkyl acetal.

The term “imino” as defined herein means a —C(═NH)— group.

The term “mercapto” as used herein means a —SH group.

The term “(NR_{A}R_{B})” as used herein means an amino group substituted by R_{A} and R_{B}. R_{A} and R_{B} are independently selected from hydrogen, alkyl, acyl, cycloalkyl, and formyl.

The term “(NR_{A}R_{B})alkyl” as used herein means an —NR_{A}R_{B} group, as defined herein, appended to the parent molecular moiety through an alkyl group, as defined herein. Representative examples of (NR_{A}R_{B})alkyl include, but are not limited to, 2-(methylamino)ethyl, 2-(dimethylamino)ethyl, 2-(amino)ethyl, 2-(ethylmethylamino)ethyl, and the like.

The term “(NR_{A}R_{B})carbonyl” as used herein means an —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, (ethylmethylamino)carbonyl, and the like.

The term “(NR_{A}R_{B})sulfonyl” as used herein means a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl and (ethylmethylamino)sulfonyl.

The term “—N(R₁₄ₐ)SO₂(R_{14b})” as used herein means an amino group attached to the parent moiety to which is further appended with an R₁₄ₐ group as defined herein, and a SO₂ group to which is appended an (R_{14b}) group as defined herein. R₁₄ₐ and R_{14b} are each independently hydrogen, alkyl, or cycloalkyl. Representative examples of —N(R₁₄ₐ)SO₂(R_{14b}) include, but are not limited to, N-methylmethanesulfonamide.

The term “SO₂N(R₁₄ₐ)(R_{14b})” as used herein means a N(R₁₄ₐ)(R_{14b}) group attached to a SO₂ group, appended to the parent moiety through the sulfonyl group. R₁₄ₐ and R_{14b} are each independently hydrogen, alkyl, or cycloalkyl. Representative examples of SO₂N(R₁₄ₐ)(R_{14b}) include, but are not limited to (dimethylamino)sulfonyl and N-cyclohexyl-N-methylsulfonyl.

The term “nitro” as used herein means a —NO₂ group.

The term “nitrogen protecting group” as used herein means those groups intended to protect a nitrogen atom against undesirable reactions during synthetic procedures. Nitrogen protecting groups comprise carbamates, amides, N-benzyl derivatives, and imine derivatives. Preferred nitrogen protecting groups are acetyl, benzoyl, benzyl, benzyloxycarbonyl (Cbz), formyl, phenylsulfonyl, pivaloyl, tert-butoxycarbonyl (Boc), tert-butylacetyl, trifluoroacetyl, and triphenylmethyl (trityl). Nitrogen-protecting groups are appended onto primary or secondary amino groups by reacting the compound that contains the amine group with base, such as triethylamine, and a reagent selected from an alkyl halide, an alkyl trifilate, a dialkyl anhydride, for example as represented by an alkyl anhydride (alkyl-OC═O)₂O, a diaryl anhydride, for example as represented by (aryl-OC═O)₂O, an acyl halide, an alkylchloroformate, or an alkylsulfonylhalide, an arylsulfonylhalide, or halo-CON(alkyl)₂, for example acetylchloride, benzoylchloride, benzylbromide, benzyloxycarbonylchloride, formylfluoride, phenylsulfonylchloride, pivaloylchloride, (tert-butyl-O—C═O)₂O, trifluoroacetic anhydride, and triphenylmethylchloride.

The term “oxo” as used herein means (═O).

The term “sulfonyl” as used herein means a —S(O)₂— group.

As used herein, the term “radiolabel” refers to a compound of the invention in which at least one of the atoms is a radioactive atom or radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

### Compounds of the Invention

Compounds of formula (I) are disclosed,

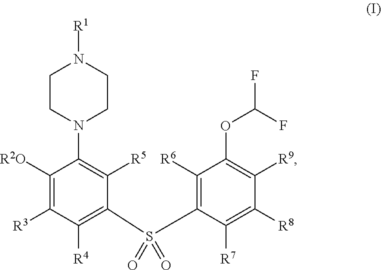

wherein R¹, R², R³, R⁴, R⁵, R⁶, R⁷, R⁸ and R⁹ are as described in the Summary of the Invention.

In certain embodiments, the compounds of formula (I) are radiolabeled.

In certain embodiments, R¹ comprises a radiolabeled substituent group. Preferably, R¹ comprises a [¹¹C]-radiolabeled or a [¹⁸F]-radiolabeled substituent group. More preferably, R¹ is selected from ¹¹CH₃, ¹¹CH₂CH₃, ¹¹CH₂CH₂CH₃, CH₂CH₂¹⁸F, and CH₂CH₂CH₂¹⁸F.

In certain embodiments, R² comprises a radiolabeled substituent group. Preferably, R² comprises a [¹¹C]-radiolabeled or a [¹⁸F]-radiolabeled substituent group. More preferably, R² is selected from ¹¹CH₃, ¹¹CH₂CH₃, ¹¹CH₂CH₂CH₃, CH₂CH₂¹⁸F, and CH₂CH₂CH₂¹⁸F.

In certain embodiments, at least one of R³, R⁴, R⁵, R⁶, R⁷, R⁸, and R⁹ comprise a radiolabeled substituent group. Preferably, at least one of R³, R⁴, R⁵, R⁶, R⁷, R⁸, and R⁹ comprise a [¹¹C]-radiolabeled or a [¹⁸F]-radiolabeled substituent group. More preferably, R³, R⁴, R⁵, R⁶, R⁷, R⁸, and R⁹ are selected from the group consisting of hydrogen and [¹⁸F]fluoro.

Specific embodiments contemplated as part of the invention also include, but are not limited to, compounds of formula (I), as defined, for example:
1-(5-(3-(difluoromethoxy)phenylsulfonyl)-2-methoxyphenyl)-4-methylpiperazine;
1-(5-(3-(difluoromethoxy)phenylsulfonyl)-2-methoxyphenyl)-4-ethylpiperazine;
1-(5-(3-(difluoromethoxy)phenylsulfonyl)-2-methoxyphenyl)-4-propylpiperazine;
1-(5-(3-(difluoromethoxy)phenylsulfonyl)-2-ethoxyphenyl)-4-methylpiperazine;
1-(5-(3-(difluoromethoxy)phenylsulfonyl)-2-propoxyphenyl)-4-methylpiperazine;
1-(5-(3-(difluoromethoxy)phenylsulfonyl)-2-methoxyphenyl)-4-(2-fluoroethyl)piperazine;
1-(5-(3-(difluoromethoxy)phenylsulfonyl)-2-methoxyphenyl)-4-(3-fluoropropyl)piperazine;
1-(5-(3-(difluoromethoxy)phenylsulfonyl)-2-(2-fluoroethoxy)phenyl)-4-methylpiperazine;
1-(5-(3-(difluoromethoxy)phenylsulfonyl)-2-(3-fluoropropoxy)phenyl)-4-methylpiperazine;
1-(5-(5-(difluoromethoxy)-2-fluorophenylsulfonyl)-2-methoxyphenyl)-4-methylpiperazine;
1-(5-(3-(difluoromethoxy)-4-fluorophenylsulfonyl)-2-methoxyphenyl)-4-methylpiperazine;
1-(5-(3-(difluoromethoxy)-4-fluorophenylsulfonyl)-2-methoxyphenyl)piperazine;
4-(3-(difluoromethoxy)-4-fluorophenylsulfonyl)-2-(4-methylpiperazin-1-yl)phenol;
1-(5-(3-(difluoromethoxy)phenylsulfonyl)-2-methoxyphenyl)-4-[¹¹C]methylpiperazine;
1-(5-(3-(difluoromethoxy)phenylsulfonyl)-2-methoxyphenyl)-4-[1-¹¹C]ethylpiperazine;
1-(5-(3-(difluoromethoxy)phenylsulfonyl)-2-methoxyphenyl)-4-[1-¹¹C]propylpiperazine;
1-(5-(3-(difluoromethoxy)phenylsulfonyl)-2-[¹¹C]methoxyphenyl)-4-methylpiperazine;
1-(5-(3-(difluoromethoxy)phenylsulfonyl)-2-[1-¹¹C]ethoxyphenyl)-4-methylpiperazine;
1-(5-(3-(difluoromethoxy)phenylsulfonyl)-2-[1-¹¹C]propoxyphenyl)-4-methylpiperazine;
1-(5-(3-(difluoromethoxy)phenylsulfonyl)-2-methoxyphenyl)-4-(2-[¹⁸F]fluoroethyl)piperazine;
1-(5-(3-(difluoromethoxy)phenylsulfonyl)-2-methoxyphenyl)-4-(3-[¹⁸F]fluoropropyl)piperazine;
1-(5-(3-(difluoromethoxy)phenylsulfonyl)-2-(2-[¹⁸F]-fluoroethoxy)phenyl)-4-methylpiperazine;
1-(5-(3-(difluoromethoxy)phenylsulfonyl)-2-(3-[¹⁸F]-fluoropropoxy)phenyl)-4-methylpiperazine;
1-(5-(5-(difluoromethoxy)-2-[¹⁸F]-fluorophenylsulfonyl)-2-methoxyphenyl)-4-methylpiperazine;
1-(5-(3-(difluoromethoxy)-4-[¹⁸F]fluorophenylsulfonyl)-2-methoxyphenyl)-4-methylpiperazine; and
1-(5-(3-(difluoromethoxy)phenylsulfonyl)-2-methoxyphenyl)-4-([³H]methyl)-piperazine.

Compounds of the invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the invention may exist as cis or trans isomers, wherein substituents on a ring may attached in such a manner that they are on the same side of the ring (cis) relative to each other, or on opposite sides of the ring relative to each other (trans). Individual cis or trans isomers of compounds of the invention may be prepared synthetically from commercially available starting materials using selective organic transformations, or by prepared in single isomeric form by purification of mixtures of the cis and trans isomers. Such methods are well-known to those of ordinary skill in the art, and may include separation of isomers by recrystallization or chromatography.

It should be understood that the compounds of the invention may possess tautomeric forms, as well as geometric isomers, and that these also constitute an aspect of the invention. It is also understood that the compounds of the invention may exist as isotopomers, wherein atoms may have different weights; for example, hydrogen and deuterium, or ¹¹C, ¹²C and ¹³C.

### Methods for Preparing Compounds of the Invention

The compounds of the invention can be better understood in connection with the following synthetic schemes and methods which illustrate a means by which the compounds can be prepared.

The compounds of this invention can be prepared by a variety of synthetic procedures. Representative procedures are shown in, but are not limited to, Schemes 1-12.

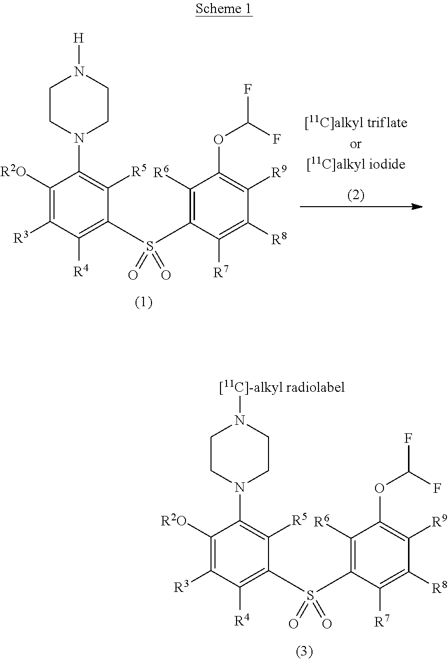

[¹¹C]-radiolabeled compounds of formula (3) can be prepared as described in Scheme 1. Compounds of formula (1), when treated with radiolabeled alkyl triflates or [¹¹C]-radiolabeled alkyl iodides of formula (2) in the presence of a base, will provide [¹¹C]-radiolabeled compounds of formula (3). For example, compounds of formula (1), when treated with [¹¹C]methyl triflate, [1-¹¹C]ethyl triflate or [1-¹¹C]n-propyl triflate, will provide, respectively, compounds of formula (3) wherein the [¹¹C]-alkyl radiolabel is ¹¹CH₃, ¹¹CH₂CH₃, or ¹¹CH₂CH₂CH₃. The [¹¹C]alkyl triflates can be prepared by methodologies known to those of ordinary skill in the art, such as for example, by passing the corresponding [¹¹C]alkyl iodides through a quartz tube loaded with silver triflate heated at 195° C. The required [¹¹C]alkyl iodides can be prepared by halogenation of the corresponding [¹¹C]alkanes in a gas phase process. For example, [¹¹C]CO₂ is produced via the ¹⁴N(p,α)¹¹C reaction by irradiation of a nitrogen target with 0.5% O₂ at a cyclotron. [¹¹C]CH₃I is prepared by catalytic reduction (Ni) of [¹¹C]CO₂ to [¹¹C]CH₄, followed by gas phase iodination with I₂ to provide [¹¹C]methyl iodide.

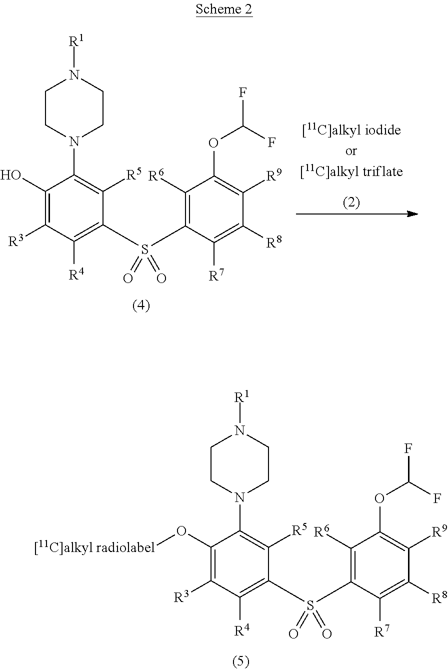

Compounds of formula (5) can be prepared as described in Scheme 2. Compounds of formula (4) when treated with [¹¹C]-radiolabeled alkyl iodides of formula (2) in the presence of a base, will provide [¹¹C]-radiolabeled compounds of formula (5). For example, compounds of formula (4), when treated with [¹¹C]methyl iodide, [1-¹¹C]ethyl iodide or [1-¹¹C]n-propyl iodide, will provide, respectively, compounds of formula (5) wherein the [¹¹C]-alkyl radiolabel is 11CH₃, ¹¹CH₂CH₃, or ¹¹CH₂CH₂CH₃.

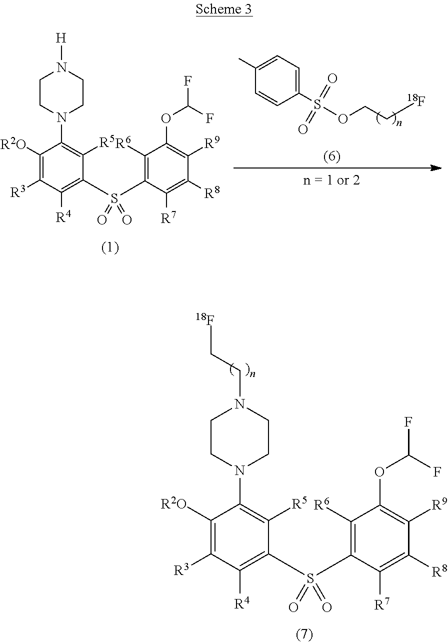

[¹⁸F]-radiolabeled compounds of formula (7) can be prepared as described in Scheme 3. Compounds of formula (1), when treated with [¹⁸F]-radiolabeled alkyl tosylates of formula (6) in the presence of a base, will provide [¹⁸F]-radiolabeled compounds of formula (7). For example, compounds of formula (1), when treated with 2-[¹⁸F]fluoroethyl tosylate or 3-[¹⁸F]fluoropropyl tosylate will provide, respectively, compounds of formula (7) wherein the [¹⁸F]-alkyl radiolabel is CH₂CH₂¹⁸F or CH₂CH₂CH₂¹⁸F. The [¹⁸F]-radiolabeled alkyl tosylates can be prepared from [¹⁸F]-fluoride generated in a cyclotron by methods known to those skilled in the art.

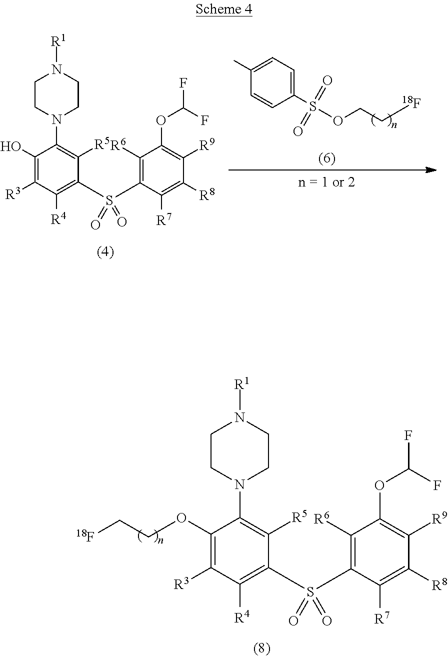

[¹⁸F]-radiolabeled compounds of formula (8) can be prepared as described in Scheme 4. Compounds of formula (4), when treated with [¹⁸F]-radiolabeled alkyl tosylates of formula (6) in the presence of a base, will provide [¹⁸F]-radiolabeled compounds of formula (8). For example, compounds of formula (4), when treated with 2-[¹⁸F]fluoroethyl tosylate or 3-[¹⁸F]fluoropropyl tosylate will provide, respectively, compounds of formula (8) wherein the [¹⁸F]-alkyl radiolabel is CH₂CH₂¹⁸F or CH₂CH₂CH₂¹⁸F.

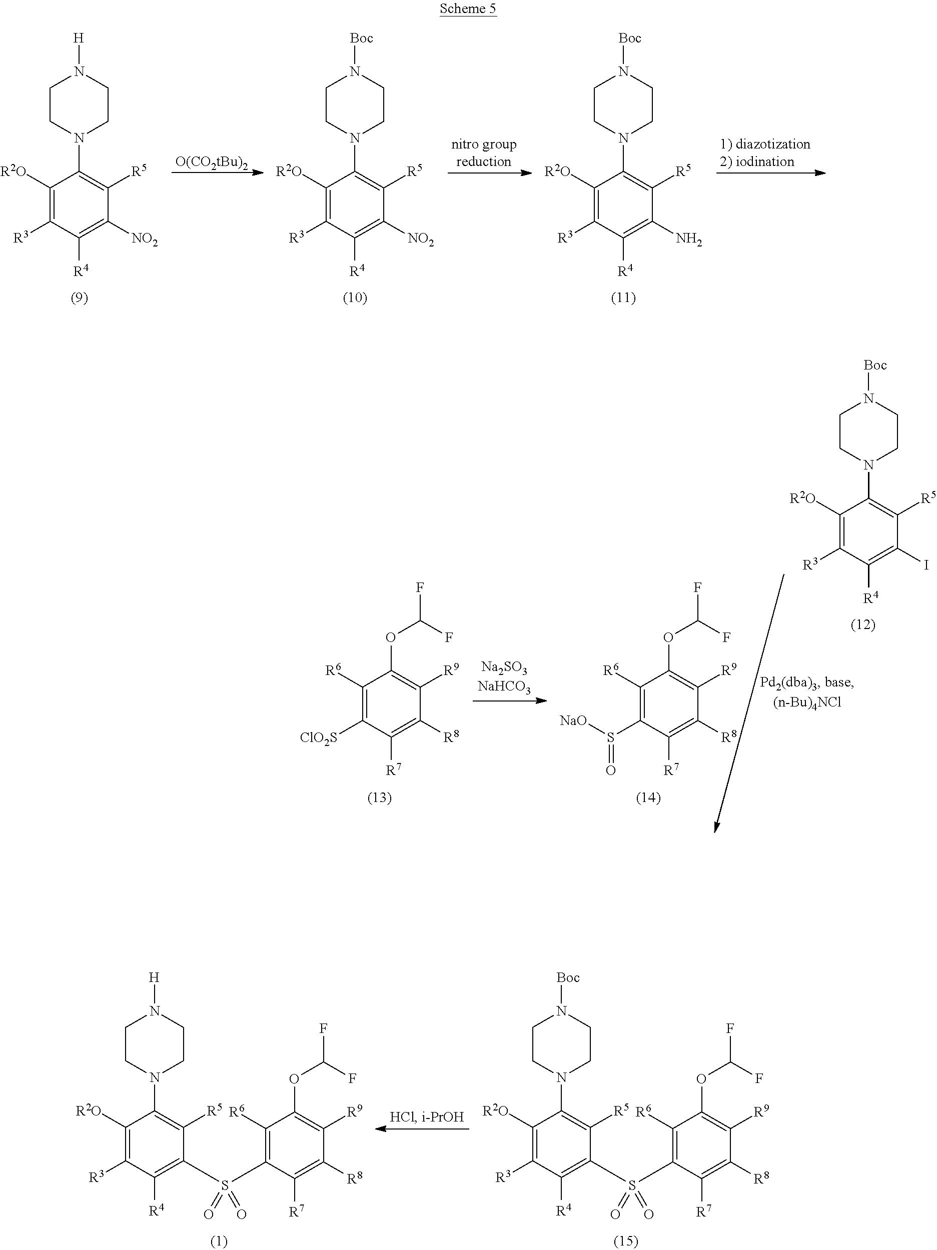

Compounds of formula (1) can be prepared as described in Scheme 5. Compounds of formula (9), which are commercially available, when treated with di-tert-butyl-dicarbonate in the presence of a base, will provide Boc-protected compounds of formula (10). The nitro group in the compounds of formula (10) can be reduced with standard aryl-nitro group reduction protocols (e.g., H₂ over Pd/C or ammonium formate with Pd/C) to provide amino compounds of formula (11). Compounds of formula (11), when treated with n-butylnitrite, will provide an intermediate diazonium salt derivative, which when treated with tetra-n-butylammonium iodide, provides iodo compounds of formula (12). The compounds of formula (12) can be coupled with compounds of formula (14) in the presence of catalytic Pd₂(dba)₃, base and tetra-n-butyl-ammonium chloride to provide compounds of formula (15). The compounds of formula (14) can be prepared from commercially available compounds of formula (13) via treatment with a mixture of sodium bicarbonate and sodium sulfite. The compounds of formula (15), when treated with acidic conditions, will provide compounds of formula (1).

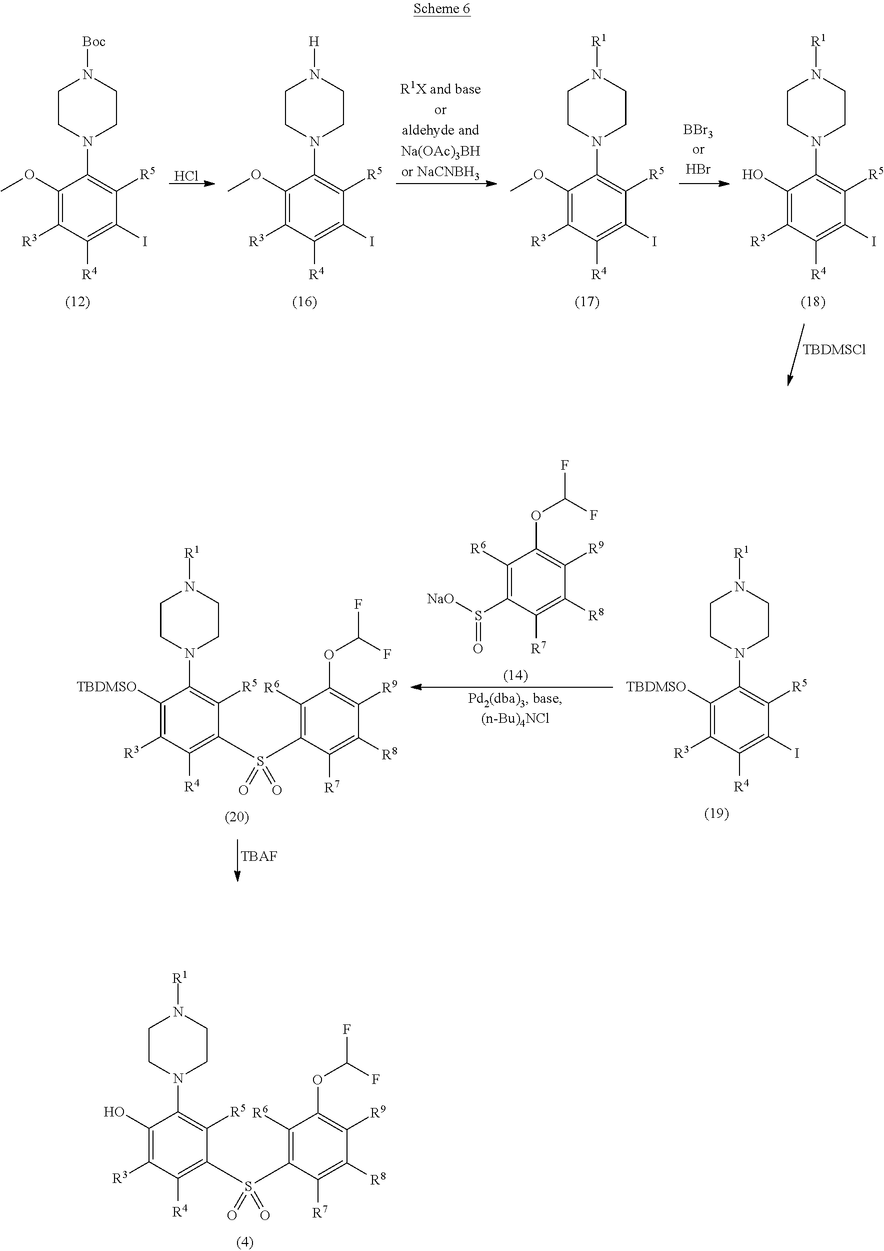

Compounds of formula (4) can be prepared as described in Scheme 6. Compounds of (12), when treated with acid (e.g., HCl in isopropanol), will provide compounds of formula (16). The unprotected nitrogen of the compounds of formula (16) can be substituted by an R¹ group (e.g., methyl) by treatment with, for example, an alkyl iodide (e.g., methyl iodide) or alternatively with an aldehyde (e.g. formaldehyde) and either NaBH(OAc)₃ or NaCNBH₃, thereby providing compounds of formula (17). Compounds of formula (17), when treated with the appropriate reagent, such as BBr₃, or HBr, or TMSI, will provide O-de-methylated compounds of formula (18). The compounds of formula (18), when treated with tert-butyldimethylsilyl chloride, will provide compounds of formula (19). The compounds of formula (19) can be coupled with compounds of formula (14) in the presence of catalytic Pd₂(dba)₃, base and tetra-n-butyl-ammonium chloride to provide compounds of formula (20). Subjecting compounds of formula (20) with tetrabutyl ammonium fluoride (TBAF) will provide compounds of formula (4).

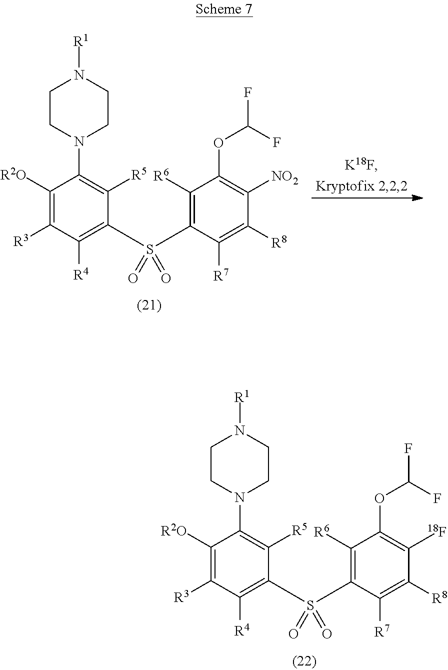

Fluorinated compounds of formula (22) can be prepared as described in Scheme 7. Compounds of formula (21), when treated with potassium [¹⁸F]fluoride in the presence of Kryptofix 2,2,2, will provide radiolabeled compounds of formula (22).

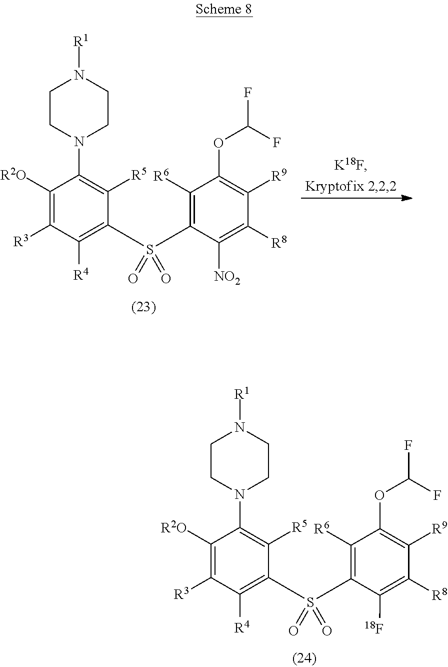

Similarly, compounds of formula (24) can be prepared as described in Scheme 8. Compounds of formula (23), when treated with potassium [¹⁸F]fluoride in the presence of Kryptofix 2,2,2, will provide radiolabeled compounds of formula (24).

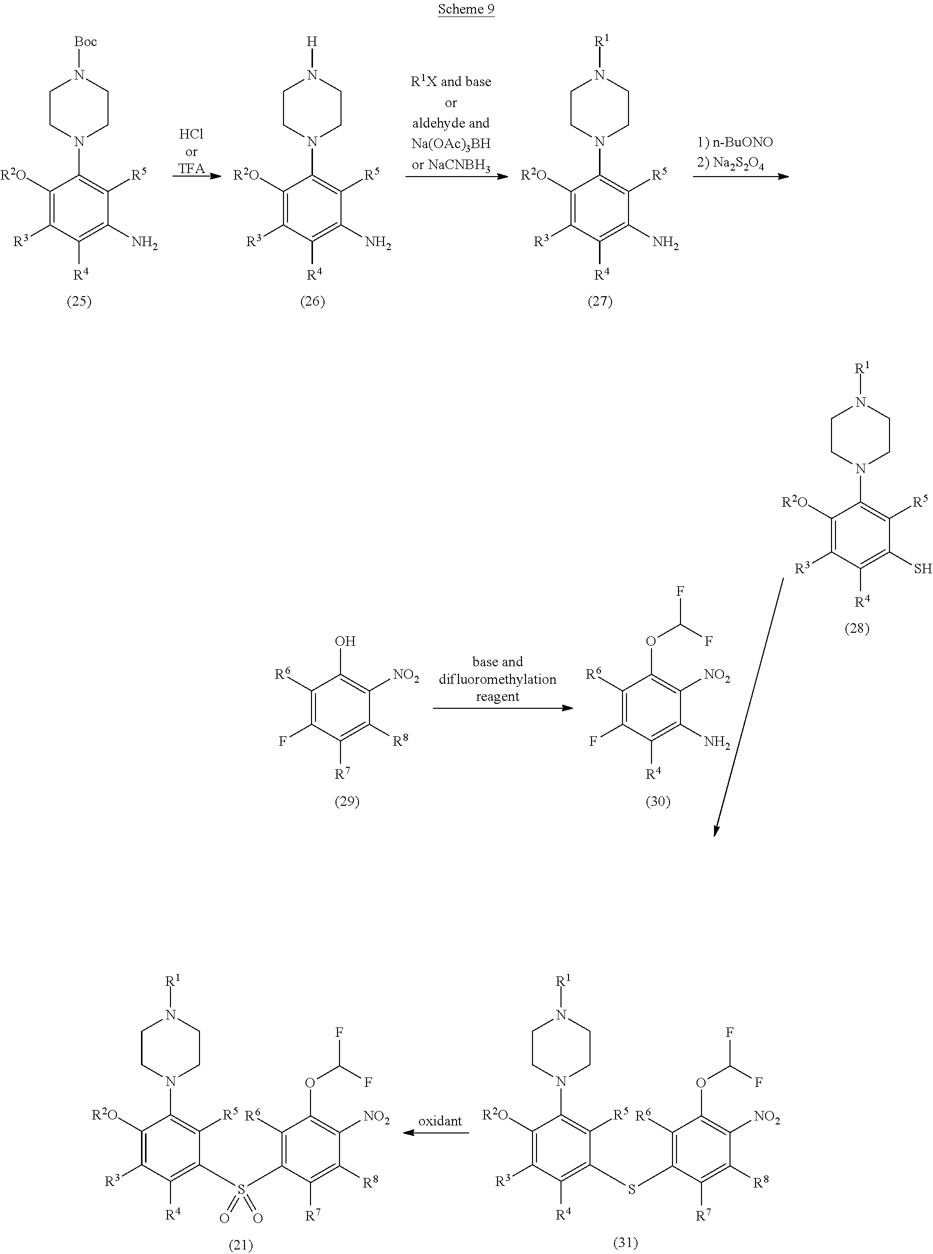

Compounds of formula (21) can be prepared as described in Scheme 9. Commercially available compounds of formula (25), when treated with acid (e.g., HCl in isopropanol), will provide deprotected compounds of formula (26). The unprotected nitrogen of the compounds of formula (26) can be substituted by an R¹ group (e.g., methyl) by treatment with, for example, an alkyl iodide (e.g., methyl iodide) or alternatively with an aldehyde (e.g. formaldehyde) and either NaBH(OAc)₃ or NaCNBH₃, thereby providing compounds of formula (27). Compounds of formula (27), when treated with n-butylnitrite, will provide intermediate diazonium salt derivatives, which when treated with sodium hydrosulfite will provide thiol compounds of formula (28). The compounds of formula (28) can be coupled with compounds of formula (30) in the presence of catalytic Pd₂(dba)₃, base and tetra-n-butyl-ammonium chloride to provide thioether compounds of formula (31). The compounds of formula (30) can be prepared by treating commercially available compounds of formula (29) with chlorodifluoromethane, difluoroiodomethane, methyl chlorodifluoroacetate, ethyl chlorodifluoroacetate, or sodium chlorodifluoroacetate in the presence of a base. The compounds of formula (31) can be oxidized with reagents such as dihydrogen peroxide, meta-chloroperbenzoic acid, sodium periodate, or oxone to provide compounds of formula (21), thereby converting the thioether functionality to a sulfone.

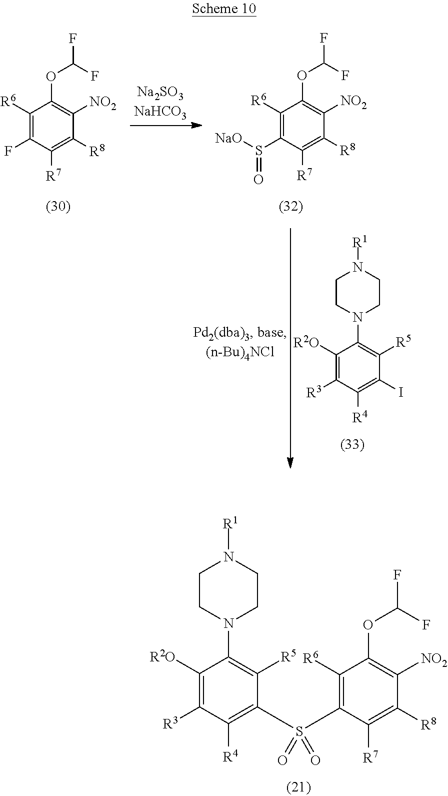

Alternatively, the compounds of formula (21) can be prepared as described in Scheme 10. Compounds of formula (30), when treated with a mixture of sodium bicarbonate and sodium sulfite, will provide compounds of formula (32). The compounds of formula (32) can be coupled with iodo compounds of formula (33) in the presence of catalytic Pd₂(dba)₃, base and tetra-n-butyl-ammonium chloride to provide compounds of formula (21).

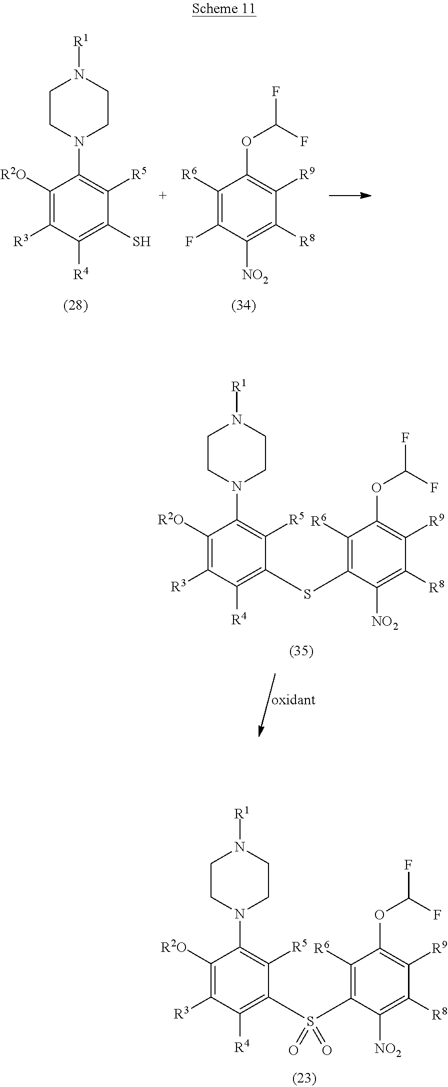

Compounds of formula (23) can be prepared as described in Scheme 11. Compounds of formula (28) can be coupled with compounds of formula (34) to provide compounds of formula (35). The compounds of formula (35) can be oxidized with reagents such as dihydrogen peroxide, meta-chloroperbenzoic acid, sodium periodate, or oxone to provide compounds of formula (23), thereby converting the thioether functionality to a sulfone.

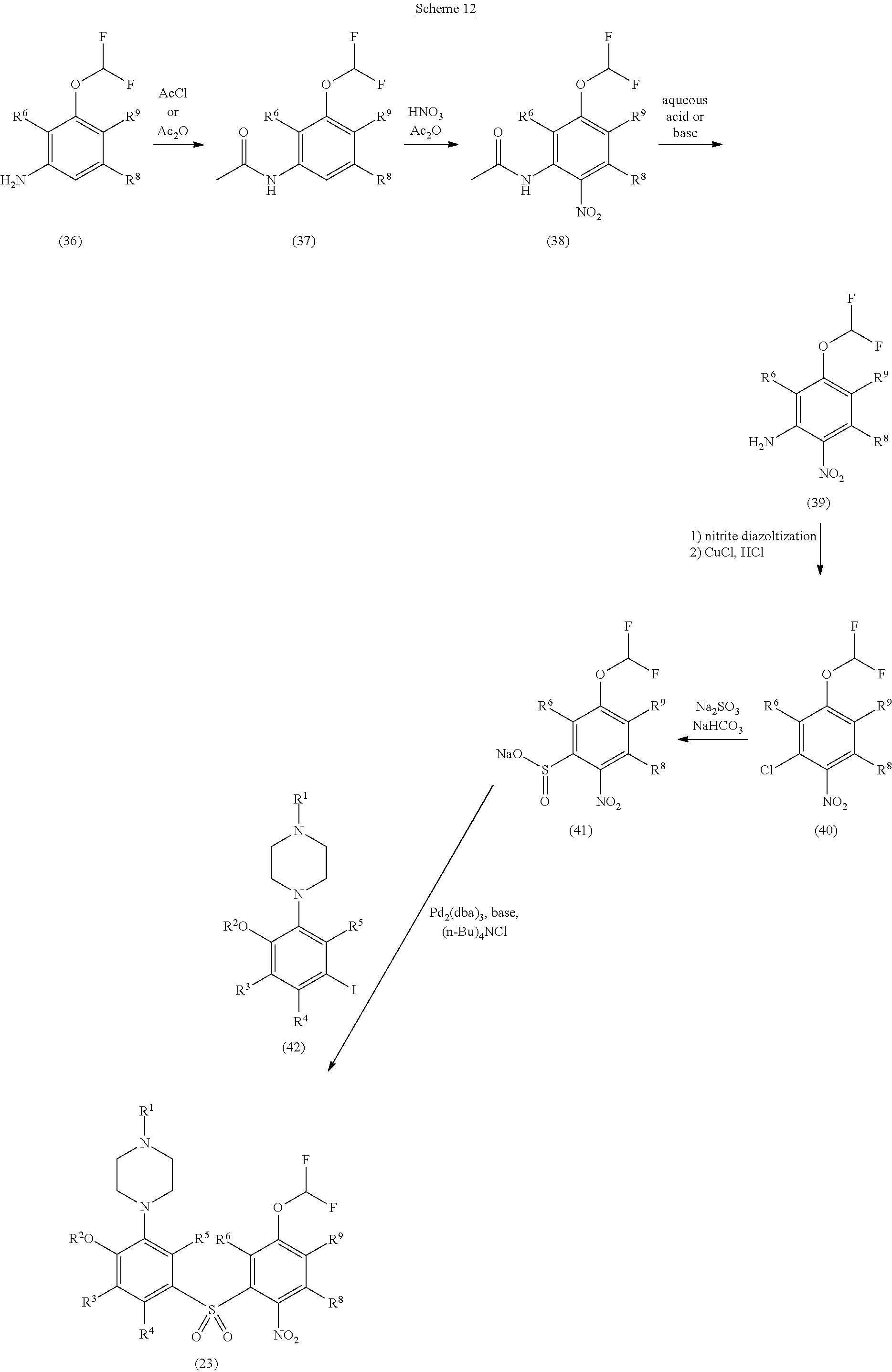

Alternatively, compounds of formula (23) can be prepared as described in Scheme 12. Compounds of formula (36), when treated with an acylating agent (e.g., acetyl chloride), will provide compounds of formula (37). Nitration of the compounds of formula (37) with HNO₃ and Ac₂O will provide compounds of formula (38). The compounds of formula (38), when treated with either strong acid (e.g. HCl) in aqueous alcohol or strong base (e.g. NaOH) in aqueous alcohol, will provide compounds of formula (39). The amino group of the compounds of formula (39) can be converted to a chloro substituent by treatment with sodium nitrite, potassium nitrite, n-butyl nitrite, or tert-butylnitrite, to provide intermediate diazonium salt derivatives, which when treated with HCl and CuCl provide compounds of formula (40). Compounds of formula (40), when treated with a mixture of sodium bicarbonate and sodium sulfite, will provide compounds of formula (41). The compounds of formula (41) can be coupled with compounds of formula (42) in the presence of catalytic Pd₂(dba)₃, base and tetra-n-butyl-ammonium chloride to provide compounds of formula (23).

Non-radiolabeled compounds of formula (I) can be prepared as described according to Schemes 1-12. For example, compounds of formula (I) wherein R¹ is (¹²C)methyl can be prepared by following the synthetic sequences of Scheme 1. Compounds of formula (I) wherein R¹ is (¹⁹F)-fluoroethyl can be prepared by following the synthetic sequences of Scheme 3.

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

Where compounds of the invention have at least one basic nitrogen, the compounds of the invention may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzensulfonic, carbonic, fumaric, maleic, gluconic, acetic, propionic, salicylic, hydrochloric, hydrobromic, phosphoric, sulfuric, citric, or hydroxybutyric acid, camphorsulfonic, malic, phenylacetic, aspartic, glutamic, and the like.

### Compositions of the Invention

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally”, as used herein, refers to modes of administration which include intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of materials which can be useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants, which can be required. Ophthalmic formulations, eye ointments, powders and solutions are contemplated as being within the scope of this invention. Aqueous liquid compositions comprising compounds of the invention also are contemplated.

The compounds of the invention can be used in the form of pharmaceutically acceptable salts, esters, or amides derived from inorganic or organic acids. The term “pharmaceutically acceptable salts, esters and amides”, as used herein, refer to carboxylate salts, amino acid addition salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Preferred salts of the compounds of the invention are the tartrate and hydrochloride salts.

Also, basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester”, as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆ alkyl esters and C₅-to-C₇ cycloalkyl esters, although C₁-to-C₄ alkyl esters are preferred. Esters of the compounds of formula (I) may be prepared according to conventional methods. For example, such esters may be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyliodide, benzyl iodide, cyclopentyl iodide. They also may be prepared by reaction of the compound with an acid such as hydrochloric acid and an alkylcarboxylic acid such as acetic acid, or with acid and an arylcarboxylic acid such as benzoic acid.

The term “pharmaceutically acceptable amide”, as used herein, refers to non-toxic amides of the invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) may be prepared according to conventional methods. Pharmaceutically acceptable amides are prepared from compounds containing primary or secondary amine groups by reaction of the compound that contains the amino group with an alkyl anhydride, aryl anhydride, acyl halide, or aryl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also may be prepared by reaction of the compound with an acid such as sulfuric acid and an alkylcarboxylic acid such as acetic acid, or with acid and an arylcarboxylic acid such as benzoic acid under dehydrating conditions as with molecular sieves added.

The compounds of the invention can be used in the form of a pharmaceutically acceptable prodrug. The pharmaceutical compositions of the invention can contain compounds of the invention in the form of a pharmaceutically acceptable prodrug.

The term “pharmaceutically acceptable prodrug” or “prodrug”, as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention may be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987), hereby incorporated by reference.

The invention contemplates pharmaceutically active compounds either chemically synthesized or formed by in vivo biotransformation to compounds of formula (I).

### Methods of the Invention

The compounds and compositions of the invention are useful for treating and preventing certain diseases and disorders in humans and animals. As an important consequence of the ability of the compounds of the invention to modulate the effects of 5-HT₆ in cells, the compounds described in the invention can affect physiological processes in humans and animals. In this way, the compounds and compositions described in the invention are useful for treating and preventing diseases and disorders modulated by 5-HT₆ receptors. Typically, treatment or prevention of such diseases and disorders can be effected by selectively modulating 5-HT₆ in a mammal, by administering a compound or composition of the invention, either alone or in combination with another active agent as part of a therapeutic regimen.

The compounds of the invention, including but not limited to those specified in the examples, possess an affinity for 5-HT₆ receptors and therefore, the compounds of the invention may be useful for the treatment and prevention of diseases or disorders of the central nervous system.

Disorders or diseases of the central nervous system are understood as meaning disorders which affect the spinal cord and, in particular, the brain. Within the meaning of the invention, the term “disorder” denotes disturbances and/or anomalies which are as a rule regarded as being pathological conditions or functions and which can manifest themselves in the form of particular signs, symptoms and/or malfunctions. While the treatment according to the invention can be directed toward individual disorders, i.e. anomalies or pathological conditions, it is also possible for several anomalies, which may be causatively linked to each other, to be combined into patterns, i.e. syndromes, which can be treated in accordance with the invention.

The disorders which can be treated in accordance with the invention are in particular disorders which respond to a modulation of the 5-HT₆ receptor. They include cognitive dysfunctions, such as a deficit in memory, cognition and learning, in particular associated with Alzheimer's disease, age-related cognitive decline and mild cognitive impairment, attention deficit disorder/hyperactivity syndrome, personality disorders, such as schizophrenia, in particular cognitive deficits related with schizophrenia, affective disorders such as depression, anxiety and obsessive compulsive disorders, motion or motor disorders such as Parkinson's disease and epilepsy, migraine, sleep disorders (including disturbances of the Circadian rhythm), feeding disorders, such as anorexia and bulimia, certain gastrointestinal disorders such as Irritable Bowl Syndrome, diseases associated with neurodegeneration, such as stroke, spinal or head trauma and head injuries, such as hydrocephalus, drug addiction and obesity.

The addiction diseases include psychic disorders and behavioral disturbances which are caused by the abuse of psychotropic substances, such as pharmaceuticals or narcotics, and also other addiction diseases, such as addiction to gaming (impulse control disorders not elsewhere classified). Examples of addictive substances are: opioids (e.g. morphine, heroin and codeine), cocaine; nicotine; alcohol; substances which interact with the GABA chloride channel complex, sedatives, hypnotics and tranquilizers, for example benzodiazepines; LSD; cannabinoids; psychomotor stimulants, such as 3,4-methylenedioxy-N-methylamphetamine (ecstasy); amphetamine and amphetamine-like substances such as methylphenidate and other stimulants including caffeine. Addictive substances which come particularly into consideration are opioids, cocaine, amphetamine or amphetamine-like substances, nicotine and alcohol.

With regard to the treatment of addiction diseases, particular preference is given to those compounds according to the invention of the formula (I) which themselves do not possess any psychotropic effect. This can also be observed in a test using rats, which, after having been administered compounds which can be used in accordance with the invention, reduce their self administration of psychotropic substances, for example cocaine.

According to another aspect of the present invention, the compounds according to the invention are suitable for treating disorders whose causes can at least partially be attributed to an anomalous activity of 5-HT₆ receptors.

According to another aspect of the present invention, the treatment is directed, in particular, toward those disorders which can be influenced, within the sense of an expedient medicinal treatment, by the binding of preferably exogeneously administered binding partners (ligands) to 5-HT₆ receptors.

The diseases which can be treated with the compounds according to the invention are frequently characterized by progressive development, i.e. the above-described conditions change over the course of time; as a rule, the severity increases and conditions may possibly merge into each other or other conditions may appear in addition to those which already exist.

The compounds of formula (I) can be used to treat a large number of signs, symptoms and/or malfunctions which are connected with the disorders of the central nervous system and, in particular, the abovementioned conditions. These signs, symptoms and/or malfunctions include, for example, a disturbed relationship to reality, lack of insight and ability to meet customary social norms or the demands made by life, changes in temperament, changes in individual drives, such as hunger, sleep, thirst, etc., and in mood, disturbances in the ability to observe and combine, changes in personality, in particular emotional lability, hallucinations, ego-disturbances, distractedness, ambivalence, autism, depersonalization and false perceptions, delusional ideas, chanting speech, lack of synkinesia, short-step gait, flexed posture of trunk and limbs, tremor, poverty of facial expression, monotonous speech, depressions, apathy, impeded spontaneity and decisiveness, impoverished association ability, anxiety, nervous agitation, stammering, social phobia, panic disturbances, withdrawal symptoms in association with dependency, maniform syndromes, states of excitation and confusion, dysphoria, dyskinetic syndromes and tic disorders, e.g. Huntington's chorea and Gilles-de-La-Tourette's syndrome, vertigo syndromes, e.g. peripheral positional, rotational and oscillatory vertigo, melancholia, hysteria, hypochondria and the like.

The compounds according to the invention are preferentially suitable for treating diseases of the central nervous system, more preferably for treating cognitive dysfunctions and in particular, for treating cognitive dysfunctions associated with schizophrenia or with Alzheimer's disease.

According to another aspect of the invention the compounds of formula (I) are particularly suitable for treating addiction diseases caused for instance by the abuse of psychotropic substances, such as pharmaceuticals, narcotics, nicotine or alcohol, including psychic disorders and behavioral disturbances related thereto.

According to another aspect of the invention the compounds of formula (I) are particularly suitable for treating nutritional disorders, such as obesity, as well as diseases related thereto, such as cardiovascular diseases, digestive diseases, respiratory diseases, cancer or type 2 diabetes.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt, ester, amide, prodrug, or radiolabeled form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

For treatment or prevention of disease, the total daily dose of the compounds of this invention administered to a human or lower animal may range from about 0.0003 to about 30 mg/kg/day. For purposes of oral administration, more preferable doses can be in the range of from about 0.0003 to about 1 mg/kg/day. If desired, the effective daily dose can be divided into multiple doses for purposes of administration; consequently, single dose compositions may contain such amounts or submultiples thereof to make up the daily dose.

Compounds and compositions of the invention also are useful as diagnostic tools. The ability of PET (positron emission tomography) and sPECT (single photon emission computed tomography) to probe the degree of receptor occupancy in humans and animals by endogenous ligands or drugs is widely recognized. This constitutes the use of PET as a biomarker to assess efficacy of pharmacological interventions with drugs. The topic and use of positron-emitting ligands for these purposes has been generally reviewed, for example in “PET ligands for assessing receptor occupancy in vivo” Burns, et al Annual Reports in Medicinal Chemistry (2001), 36, 267-276; “Ligand-receptor interactions as studied by PET: implications for drug development” by Jarmo Hietala, Annals of Medicine (Helsinki) (1999), 31(6), 438-443; “Positron emission tomography neuroreceptor imaging as a tool in drug discovery, research and development” Burns, et al. Current Opinion in Chemical Biology (1999), 3(4), 388-394. The compounds of the invention, synthesized with ¹¹C, ¹⁸F, or other positron-emitting isotopes are suitable ligand tools for PET; a number of positron-emitting reagents have been synthesized, are available, and are known to those skilled in the art. Especially suitable compounds of the invention for this use are those wherein a ¹¹CH₃ group can be incorporated in by reaction with ¹¹CH₃I or CH₃OTf. Also, especially suitable compounds of the use are those wherein a ¹⁸F group can be incorporated into the compound by reaction with ¹⁸F-fluoride anion. The incorporation of ¹¹CH₃ can be carried out according to a method such as that described in Example 2. In a like manner, other compounds of formula (I) can be prepared for use in PET studies. The incorporation of ¹⁸F can be carried out according to a method such as that described in Example 4. In a like manner, other compounds of formula (I) can be prepared for use in PET studies. Among compounds of the invention that are suitable for use as ligands for PET studies are ¹⁸F and ₁₁C isotopes of compounds of the invention, including, but not limited to
1-(5-(3-(difluoromethoxy)phenylsulfonyl)-2-methoxyphenyl)-4-[¹¹C]methylpiperazine;
1-(5-(3-(difluoromethoxy)phenylsulfonyl)-2-methoxyphenyl)-4-[1-¹¹C]ethylpiperazine;
1-(5-(3-(difluoromethoxy)phenylsulfonyl)-2-methoxyphenyl)-4-[1-¹¹C]propylpiperazine;
1-(5-(3-(difluoromethoxy)phenylsulfonyl)-2-[¹¹C]methoxyphenyl)-4-methylpiperazine;
1-(5-(3-(difluoromethoxy)phenylsulfonyl)-2-[1-¹¹C]ethoxyphenyl)-4-methylpiperazine;
1-(5-(3-(difluoromethoxy)phenylsulfonyl)-2-[1-¹¹C]propoxyphenyl)-4-methylpiperazine;
1-(5-(3-(difluoromethoxy)phenylsulfonyl)-2-methoxyphenyl)-4-(2-[¹⁸F]fluoroethyl)piperazine;
1-(5-(3-(difluoromethoxy)phenylsulfonyl)-2-methoxyphenyl)-4-(3-[¹⁸F]fluoropropyl)piperazine;
1-(5-(3-(difluoromethoxy)phenylsulfonyl)-2-(2-[¹⁸F]-fluoroethoxy)phenyl)-4-methylpiperazine;
1-(5-(3-(difluoromethoxy)phenylsulfonyl)-2-(3-[¹⁸F]-fluoropropoxy)phenyl)-4-methylpiperazine;
1-(5-(5-(difluoromethoxy)-2-[¹⁸F]-fluorophenylsulfonyl)-2-methoxyphenyl)-4-methylpiperazine;
1-(5-(3-(difluoromethoxy)-4-[¹⁸F]-fluorophenylsulfonyl)-2-methoxyphenyl)-4-methylpiperazine; and
1-(5-(3-(difluoromethoxy)phenylsulfonyl)-2-methoxyphenyl)-4-([³H]methyl)-piperazine.

The compounds and processes of the invention will be better understood by reference to the following examples, which are intended as an illustration of and not a limitation upon the scope of the invention.

### EXAMPLES

### Intermediate 1

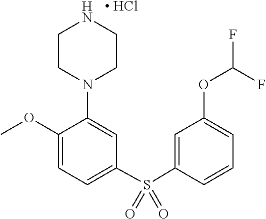 [SMILES: "· HCl" ; COc1ccc(S(=O)(=O)c2cccc(OC(F)F)c2)cc1N1CCNCC1]

### 4-(5-(3-(difluoromethoxy)phenylsulfonyl)-2-methoxyphenyl)piperazin-1-ium chloride

### Intermediate 1A

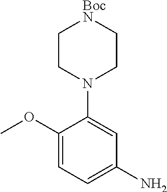 [SMILES: COc1ccc(N)cc1N1CCN(C(=O)OC(C)(C)C)CC1]

### tert-butyl 4-(5-amino-2-methoxyphenyl)piperazine-1-carboxylate

tert-Butyl 4-(2-methoxy-5-nitrophenyl)piperazine-1-carboxylate (CAS#177488-99-2, Fluorochem #035927, 4 g, 11.86 mmol) was added to a flask containing ethanol (30 mL), distilled water (5 mL), and 10% Pd on activated carbon (0.25 g). The reaction mixture was stirred and heated to 80° C. A solution of ammonium formate (7.48 g, 119 mmol) in distilled water (5 mL) was added dropwise and the reaction mixture was stirred for 1 hour. TLC (10:1 toluene/MeOH in ammonia saturated atmosphere) indicated quantitative formation of a new product. The reaction mixture was filtered and the filtrate was concentrated under reduced pressure. The residue was partitioned between CH₂Cl₂ and distilled water. The organic phase was separated and the aqueous phase was extracted twice with CH₂Cl₂. The combined organic phases were dried (MgSO₄) to give a quantitative amount of tert-butyl 4-(5-amino-2-methoxyphenyl)piperazine-1-carboxylate as a yellow-brown resin. This resin was used in the next step without further purification. LC/MS (APCI), m/z=308.2 (M+H)⁺.

### Intermediate 1B

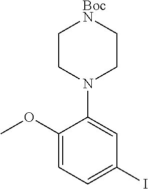 [SMILES: COc1ccc(I)cc1N1CCN(C(=O)OC(C)(C)C)CC1]

### tert-butyl 4-(5-iodo-2-methoxyphenyl)piperazine-1-carboxylate

Intermediate 1A, tert-butyl 4-(5-amino-2-methoxyphenyl)piperazine-1-carboxylate (318 mg, 1.035 mmol) was dissolved in a small amount of CH₂Cl₂ and cooled to 0° C. TFA (1 equivalent), diluted in CH₂Cl₂, was added dropwise and then the reaction was evaporated in an air bath. The residue was dissolved in acetonitrile (2.5 mL) and the resulting solution was added dropwise to a stirred solution of n-butylnitrite in acetonitrile (7.5 mL) at 0° C. After stirring 10 minutes, tetra-n-butylammonium iodide (764 mg, 2.069 mmol) was added in portions at 0 to +5° C. The reaction mixture was stirred for 2 hours at 0° C. TLC (10:1 toluene/MeOH in ammonia saturated atmosphere) indicated total conversion of the starting material to a product with massive impurities. The reaction mixture was concentrated under reduced pressure and the residue was dissolved in CH₂Cl₂. The solution was washed twice with 10% aqueous sodium thiosulfate, dried (MgSO₄), and concentrated under reduced pressure. The residue was purified by chromatography using a mixture of n-heptane and ethyl acetate to give tert-butyl 4-(5-iodo-2-methoxyphenyl)piperazine-1-carboxylate as a yellow resin which was used in the next step without further purification (88 mg, 20.3%). LC/MS: (APCI) m/z=419.1 (M+H)⁺.

### Intermediate 1C

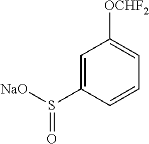

### sodium 3-(difluoromethoxy)benzenesulfinate

A stirred mixture of sodium bicarbonate (3.39 g, 40.4 mmol) and sodium sulfite (2.55 g, 20.2 mmol) in distilled water (15 mL), was heated to 75° C. 3-(Difluoromethoxy)benzene-1-sulfonyl chloride (CAS#351003-38-8, Aldrich #554405, 4.90 g) was added dropwise over 30 minutes, then the reaction mixture was stirred at 80° C. for 5 hours. TLC (2:1 n-heptane/ethyl acetate) indicated total conversion of the sulfonylchloride to a very polar product, located at the origin. The reaction mixture was concentrated under reduced pressure and co-distilled with toluene. The solid, dry residue was stirred with methanol (25 mL), then filtered. The filter cake was washed with a small amount of methanol. The combined filtrates were evaporated to give a quantitative yield of sodium 3-(difluoromethoxy)benzenesulfinate as yellowish crystals. LC/MS: (APCI) m/z=209.0 (M+H)⁺.

### Intermediate 1D

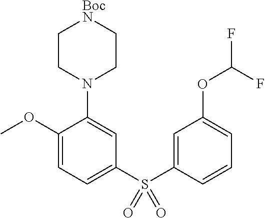 [SMILES: COc1ccc(S(=O)(=O)c2cccc(OC(F)F)c2)cc1N1CCN(C(=O)OC(C)(C)C)CC1]

### tert-butyl 4-(5-(3-(difluoromethoxy)phenylsulfonyl)-2-methoxyphenyl)piperazine-1-carboxylate

A mixture of Intermediate 1B, tert-butyl 4-(5-iodo-2-methoxyphenyl)piperazine-1-carboxylate (287 mg, 0.686 mmol), Intermediate 1C, sodium 3-(difluoromethoxy)-benzenesulfinate (190 mg, 0.823 mmol), Cs₂CO₃ (335 mg, 1.029 mmol), tetra-n-butyl-ammonium chloride (229 mg, 0.823 mmol), Xantphos (20 mg, 0.034 mmol) and Pd₂(dba)₃ (16 mg, 0.017 mmol) in toluene (10 mL) were stirred under nitrogen at 80° C. for 8 hours. TLC (2:1 n-heptane/ethyl acetate) indicated total conversion of the starting materials to an impure product. The reaction mixture was filtered and the filtrate was concentrated under reduced pressure. The residue was purified by chromatography using 2:1 n-heptane/ethyl acetate to provide tert-butyl 4-(5-(3-(difluoromethoxy)phenylsulfonyl)-2-methoxyphenyl) piperazine-1-carboxylate as a red resin which was used in the next step without further purification (241 mg, 70.5%). LC/MS: (APCI) m/z=499.20 (M+H)⁺.

### Intermediate 1

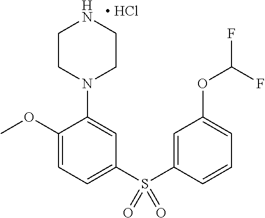 [SMILES: "· HCl" ; COc1ccc(S(=O)(=O)c2cccc(OC(F)F)c2)cc1N1CCNCC1]

### 4-(5-(3-(difluoromethoxy)phenylsulfonyl)-2-methoxyphenyl)piperazin-1-ium chloride

Intermediate 1D, tert-butyl 4-(5-(3-(difluoromethoxy)phenylsulfonyl)-2-methoxyphenyl) piperazine-1-carboxylate (241 mg, 0.483 mmol) was dissolved in ethanol (5 mL) and 5-6M HCl in 2-propanol (5 mL) was added. The solution stirred overnight at ambient temperature. TLC showed total conversion of the starting material to a slightly impure product. The reaction mixture was concentrated under reduced pressure. The residue was dissolved in a small amount of ethanol and crystallization was induced with diethyl ether. The product was recrystallized from 2-propanol to give a quantitative amount of 4-(5-(3-(difluoromethoxy)phenylsulfonyl)-2-methoxyphenyl)piperazin-1-ium chloride. ¹H-NMR (500 MHz, DMSO-d₆): δ 3.16 (s, 4H), 3.25 (s, 4H), 3.87 (s, 3H), 7.18 (d, 1H), 7.24-7.54 (t, 1H), 7.39 (s, 1H), 7.46 (d, 1H), 7.65 (t+d, 2H), 7.73 (s, 1H), 7.82 (d, 1H), 9.52 (s, 2H). LC/MS: (APCI) m/z=399.1 (M+H)⁺.

Alternatively, Intermediate 1 can be prepared from commercially available starting materials 5-iodo-methoxyaniline and N,N-bis(2-chloroethyl)-4-methylbenzenesulfonamide. Treatment of 5-iodo-methoxyaniline and N,N-bis(2-chloroethyl)-4-methylbenzenesulfonamide with sodium carbonate and potassium iodide in cyclohexanol at 160° C. will provide 1-(5-iodo-2-methoxyphenyl)-4-tosylpiperazine. Coupling of 1-(5-iodo-2-methoxyphenyl)-4-tosylpiperazine with sodium 3-(difluoromethoxy)benzenesulfinate can be accomplished by treatment with Xantphos, cesium carbonate, and tetrabutyl ammonium chloride. The resulting 1-(5-(3-(difluoromethoxy)phenylsulfonyl)-2-methoxyphenyl)-4-tosylpiperazine can be converted to Intermediate 1 by treatment with concentrated sulfuric acid at 50° C. for 5 hours.

### Example 1

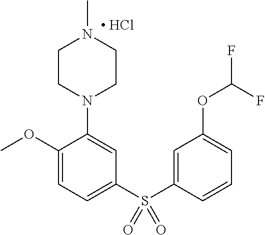 [SMILES: COc1ccc(S(=O)(=O)c2cccc(OC(F)F)c2)cc1N1CCN(C)CC1 ; "· HCl"]

### 4-(5-(3-(difluoromethoxy)phenylsulfonyl)-2-methoxyphenyl)-1-methylpiperazin-1-ium chloride

The free base of Intermediate 1 was prepared by dissolving the HCl salt (122 mg, 0.281 mmol) in a solution of NaOH (11.24 mg, 0.281 mmol) in methanol (10 mL) then concentrating the solution under reduced pressure. The residue was treated with CH₂Cl₂ (5 mL), followed by acetic acid (17 mg, 0.281 mmol) and 37% aqueous formaldehyde solution (8.42 mg, 0.281 mmol), along with sodium triacetoxyborohydride (59 mg, 0.281 mmol). The reaction mixture was stirred at ambient temperature for 18 hours. TLC (10:1 toluene/MeOH in ammonia saturated atmosphere) indicated clean conversion to one product. The reaction mixture was diluted with CH₂Cl₂ and washed with aqueous sodium bicarbonate. The organic phase was separated and the aqueous phase was extracted twice with CH₂Cl₂. The combined organic phases were dried (MgSO₄) and filtered. The filtrate was concentrated under reduced pressure. The residue was dissolved in THF and a solution of 2M hydrochloric acid in diethyl ether was added. The crystallized salt was collected by filtration, washed with ether, and dried in vacuo to provide 4-(5-(3-(difluoromethoxy)phenylsulfonyl)-2-methoxyphenyl)-1-methylpiperazin-1-ium chloride as a light beige solid (89 mg, 70.7%). ¹H-NMR (500 MHz, DMSO-d₆): δ 2.79 (s, 3H), 3.04-3.25 (m, 4H), 3.45 (d, 2H), 3.53 (d, 2H), 3.86 (s, 3H), 7.19 (d, 1H), 7.24-7.54 (t+s, 2H), 7.46 (d, 1H), 7.65 (t+d, 2H), 7.75 (s, 1H), 7.82 (d, 1H). LC/MS: (APCI) m/z=413.1 (M+H)⁺.

Free base (1-(5-(3-(difluoromethoxy)phenylsulfonyl)-2-methoxyphenyl)-4-methylpiperazine): ¹H NMR (300 MHz, methanol-d₄) δ 7.79-7.74 (m, 1H), 7.69-7.55 (m, 3H), 7.43-7.35 (m, 2H), 7.12 (d, J=8.6, 1H), 6.92 (td, J=73 and 0.5, 1H), 3.92 (s, 3H), 3.16-3.00 (m, 4H), 2.70-2.56 (m, 4H), 2.35 (s, 3H).

### Example 2

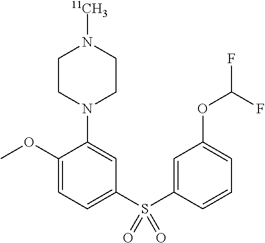 [SMILES: COc1ccc(S(=O)(=O)c2cccc(OC(F)F)c2)cc1N1CCN([11CH3])CC1]

### 1-(5-(3-(difluoromethoxy)phenylsulfonyl)-2-methoxyphenyl)-4-([¹¹C]methyl)-piperazine

[¹¹C]CO₂ is produced via the ¹⁴N(p,α)¹¹C reaction by irradiation of a nitrogen target with 0.5% O₂ at a cyclotron. [¹¹C]CH₃I is prepared by catalytic reduction (Ni) of [¹¹C]CO₂ to [¹¹C]CH₄, followed by gas phase iodination with I₂. Subsequently, [¹¹C]CH₃I is passed through a quartz tube loaded with silver triflate heated at 195° C. for conversion to [¹¹C]CH₃OTf. The [¹¹C]MeOTf is trapped at ambient temperature into a 1 mL glass container loaded with Intermediate 1 (free base, 1 mg) and 2,2,6,6-tetramethylpiperidine (10 μL) in 2:1 MeOH/acetonitrile (300 μL). After trapping, the reaction mixture is heated at 80° C. for 5 min then injected onto a semi-preparative column for purification. Example 2 is purified on a C18 column and the product fraction is collected and evaporated to dryness, then reformulated in dilute aqueous NaCl.

### Example 3

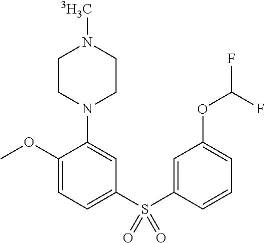

### 1-(5-(3-(difluoromethoxy)phenylsulfonyl)-2-methoxyphenyl)-4-([³H]methyl)-piperazine

A 5 mL vial was charged with [³H]methyl nosylate (1.5 mL of a solution in 20% ethyl acetate in hexanes). The mixture was carefully concentrated on a rotary evaporator. The residue obtained was treated with Intermediate 1,4-(5-(3-(difluoromethoxy)-phenylsulfonyl)-2-methoxyphenyl)piperazin-1-ium chloride (1.82 mg) and acetonitrile (1 mL) to give a turbid solution. DIEA (3 μL) was added to give a clear solution. The reaction mixture was stirred overnight at ambient temperature. The pale yellow colored solution obtained was analyzed by TLC (silica gel, 15% methanol in DCM and 0.1% NH₄OH, Rf˜0.8) and showed 86% product+14% unreacted [³H]-methyl nosylate. The reaction was continued for 24 hours. TLC (silica gel, 15% methanol in DCM and 0.1% NH₄OH, Rf˜0.8) showed 93% product+6% unreacted [³H]-methyl nosylate. At this point, the reaction was stopped and was purified by using a 1500μ silica gel prep plate (15% methanol in DCM and 0.1% NH₄OH). The band corresponding to the product was scraped and was extracted with ethyl acetate (4×25 mL) and a small amount of methanol. The combined organic extracts gave 15 mCi of Example 3,1-(5-(3-(difluoromethoxy)phenylsulfonyl)-2-methoxyphenyl)-4-([³H]methyl)-piperazine. HPLC analysis (of this ethyl acetate solution) showed a slight shoulder along with the expected radiochemical peak. The solvent was removed on a rotary evaporator and the residue obtained was dissolved in ethanol. HPLC analysis of the ethanol solution showed 2 peaks. The crude material (from the ethanol solution) was concentrated under reduced pressure and the residue was dissolved in 700 μL of 75% acetonitrile in 0.1% TFA in water. This solution was purified on prep HPLC by using isocratic 30% acetonitrile:0.1% TFA in water. Four injections were made. The product peaks were collected and purity was checked by HPLC. Two fractions containing ˜2.4 mCi showed ˜88% RCP. Another fraction containing 700 μCi showed RCP>99%. The fractions were concentrated under reduced pressure and the residue was redissolved in ethanol without lowering the purity of the desired product. Sample sitting in the autosampler overnight did not show any decomposition of desired product. MS (+ESI) m/z=419 (M+H)⁺.

### Example 4

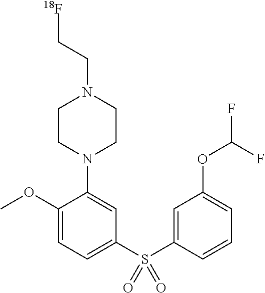 [SMILES: COc1ccc(S(=O)(=O)c2cccc(OC(F)F)c2)cc1N1CCN(CC[18F])CC1]

### 1-(5-(3-(difluoromethoxy)phenylsulfonyl)-2-methoxyphenyl)-4-(2-[¹⁸F]fluoroethyl)piperazine

### Example 4A

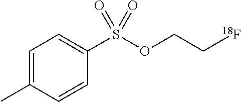

### 2-[¹⁸F]-fluoroethyl 4-methylbenzenesulfonate

To a dried Kryptofix 2.2.2./[¹⁸F]fluoride complex, 4 mg of ethyleneglycol-1,2-ditosylate in 1 mL of acetonitrile is added and heated under stirring in a sealed vial for 3 min. Purification of the crude product was accomplished using HPLC (Lichrosphere RP18-EC5, 250×10 mm, acetonitrile/water 50:50, flow rate 5 mL/min, Rf: 8 min). After diluting the HPLC fraction containing the 2-[¹⁸F]fluoroethyl 4-methylbenzenesulfonate with water (HPLC fraction/water 1:4), the product is loaded on a C18-Sepac cartridge, dried with a nitrogen stream and eluted with 1.2 mL of DMSO. The whole preparation time is about 40 min and the overall radiochemical yield is between 60 and 80%.

### Example 4

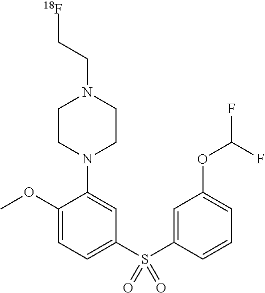 [SMILES: COc1ccc(S(=O)(=O)c2cccc(OC(F)F)c2)cc1N1CCN(CC[18F])CC1]

### 1-(5-(3-(difluoromethoxy)phenylsulfonyl)-2-methoxyphenyl)-4-(2-[¹⁸F]fluoroethyl)piperazine

To a standard Pyrex glass microwave reaction vessel containing the free base of Intermediate 1 (˜0.01 mmol), and a base such as sodium bicarbonate (1 equiv to substrate) is added a solution of 2-[¹⁸F]fluoroethyl 4-methylbenzenesulfonate in acetonitrile (radioactivity R1=0.2-2 mCi; 200-300 μL). The contents are crimp sealed with a PTFE-coated septum and irradiated in a single mode microwave cavity for ˜10 minutes at 150° C. and a power setting of 300 W. At the end of the microwave heating, the reaction mixture is cooled to room temperature, transferred out of the reaction vessel and purified by reverse phase HPLC.

Alternatively, Example 4 may be prepared by a non-microwave method, similar to that found in J. Label. Compd. Radiopharm. 2003; 46, 645-659. A solution of Intermediate 1 (6.3 μmol) in DMSO (0.5 mL) is tempered for 5 min at 140° C. Then, a solution of Example 4A, 2-[¹⁸F]fluoroethyl 4-methylbenzenesulfonate (480-560 MBq) its DMSO (500 μL) is added and stirred in a sealed reaction vessel at 140° C. for 25 min. The product is purified by reverse phase HPLC.

### Determination of Biological Activity

To determine the effectiveness of representative compounds of this invention as 5-HT₆ ligands, the following tests were conducted.

### 1. Preparation of Membranes by Ultrasonic Treatment and Differential Centrifugation

Cells from stable clonal cell lines expressing the corresponding receptor (5-HT₆, 5-HT_{2A} or 5-HT_{2B} receptors) were washed with PBS with 0.02% EDTA. The cells were collected by centrifugation at 500 g for 10 min. at 4° C., washed with PBS and centrifuged (500 g, 10 min. 4° C.). The pellets were stored at −80° C. until use. For membrane preparation, the thawed cell pellet was resuspended in ice-cold sucrose buffer (0.25 M sucrose, 10 mM Hepes (pH 7.4), 1 mM Phenylmethylsulfonyl fluoride (PMSF) in DMSO, 5 g/ml Pepstatin-A, 3 mM EDTA, 0.025% Bacitracin) and homogenized with a Branson Sonifier W-250 (Settings: Timer 4; Output Control 3; Duty Cycle constant; 2 to 3 cycles). Cell disruption was checked with the aid of a microscope. Remaining unbroken cells were pelleted at 1,000 g for 10 min. at 4° C. The sucrose buffer supernatant was then centrifuged at 60,000 g for 1 h at 4° C. (Beckman Ultrazentrifuge XL 80). The pellet was resuspended in 30 ml ice-cold Tris buffer (20 mM TRIS (pH 7.4), 5 g/ml Pepstatin A, 0.1 mM PMSF, 3 mM EDTA) by pipetting through a 10 ml serological pipet and centrifuged for 1 h at 4° C. at 60,000 g. A final resuspension was performed in a small volume of ice-cold Tris buffer (see above) by pressing through a serological pipet followed by ultrasonic treatment with a Branson Sonifier W-250 (Settings: Timer 1; Output Control 3; Duty Cycle constant; 1 cycle). Protein concentration was determined (BCA-Kit; Pierce) and aliquots stored at −80° C. or in liquid nitrogen for long-term storage.

### 2. Receptor Binding Experiments

All receptor binding experiments were carried out in the corresponding assay buffer in a total volume of 200 μl in the presence of various concentrations of test compound (10 ⁻⁵ M to 10⁻⁹ M, tenfold serial dilution, duplicate determinations). The assays were terminated by filtration on polyethylenimine (PEI 0.1% or 0.3%) presoaked Packard Unifilter Plates (GF/C or GF/B) with a Tomtec Mach III U 96 well-plate harvester. After the plates had been dried for 2 h at 55° C. in a drying chamber scintillation cocktail (BetaPlate Scint; PerkinElmer) was added. Radioactivity was measured in a Microbeta Trilux two hours after the addition of the scintillation mixture. Data derived from liquid scintillation counting were analysed by iterative non-linear regression analysis with the use of the Statistical Analysis System (SAS): a program similar to “LIGAND” as described by Munson and Rodbard (Analytical Biochemistry 107, 220-239 (1980).

### a) 5-HT₆ Receptor Binding Assay

HEK293 cells stably expressing the h-5-HT₆ receptor (NCBI Reference Sequence XM 001435) were cultured in RPMI1640 medium supplemented with 25 mM HEPES, 10% fetal calf serum and 1-2 mM glutamine. The membrane preparation was performed as described in section 1. For these membranes a K_{D} of 1.95 nM for [³H]-LSD (Lysergic Acid Diethylamide; Amersham, TRK1038) was determined by means of saturation binding experiments. On the day of the assay, the membranes were thawed, diluted in assay buffer (50 mM Tris-HCl, 5 mM CaCl₂, 0.1% ascorbic acid, 10 μM pargyline, pH 7.4) to a concentration of 8 μg protein/assay and homogenized by gentle vortexing. For inhibition studies, 1 nM [³H]-Lysergic Acid Diethylamide was incubated in the presence of various concentrations of test compound in assay buffer. Non-specific binding was defined with 1 μM methiothepin. The binding reaction was carried out for 3.5 h at room temperature. During the incubation, the plates were shaken on a plate shaker at 100 rpm and terminated by filtration on Packard Unifilter GF/C (0.1% PEI) plates, followed by 2 wash cycles with ice-cold 50 mM Tris-HCl, 5 mM CaCl₂.

### b) 5-HT_{2A} Receptor Binding Assay

CHO-K1 cell membranes expressing h-5-HT_{2A} receptor (Perkin Elmer ES-3,3-M400UA) were thawed, diluted in assay buffer (50 mM Tris-HCl, 5 mM MgCl₂, 1 mM EGTA, pH 7.4) to a final membrane protein concentration of 25 μg/ml and homogenized by gentle vortexing. For the competition binding studies; 20 μl of varying concentrations of test compounds in assay buffer (or 20 μl of assay buffer), 80 μl of 0.1 nM [¹²⁵I]-R-(−)-DOI (2,5-dimethoxy-4-iodo-amphetamine) (Perkin Elmer NEX-255) were incubated with 100 μl of homogenized membranes (total volume 200 μl). Non-specific binding was determined in the presence of 10 μM (±)-DOI (Sigma D-101). After a 1 hr incubation at room temperature, the binding reaction was harvested (Tomtec Mach III U Harvester) through 96-well GF/C filter plates (Perkin Elmer) presoaked for 1 hr with 20 μl per well of 0.3% polyethylene-imine (PEI). Harvested plates were washed twice with ice-cold assay buffer and dried prior to addition of 35 μl scintillator (BetaplateScint, Perkin Elmer). The radioactivity was determined by liquid scintillation spectrometry in a MicroBeta (Perkin Elmer) plate counter.

### c) 5-HT_{2B} Receptor Binding Assay

CHO-K1 cell membranes expressing h-5-HT_{2B} receptor (Perkin Elmer ES-3,4-M400UA) were thawed, diluted in assay buffer (50 mM Tris-HCl, 5 mM CaCl₂, pH 7.4) to a final membrane protein concentration of 25 μg/ml and homogenized by gentle vortexing. For the competition binding studies; 20 μl of varying concentrations of test compounds in assay buffer (or 20 μl of assay buffer), 80 μl of 0.1 nM [¹²⁵I]-R-(−)-DOI (2,5-dimethoxy-4-iodo-amphetamine) (Perkin Elmer NEX-255) were incubated with 100 μl of homogenized membranes (total volume 200 μl). Non-specific binding was determined in the presence of 10 μM (±)-DOI (Sigma D-101). After a 1 hr incubation at room temperature, the binding reaction was harvested (Tomtec Mach III U Harvester) through 96-well GF/C filter plates (Perkin Elmer) presoaked for 1 hr with 20 μl per well of 0.3% polyethylene-imine (PEI). Harvested plates were washed twice with ice-cold assay buffer and dried prior to addition of 35 μl scintillator (BetaplateScint, Perkin Elmer). The radioactivity was determined by liquid scintillation spectrometry in a MicroBeta (Perkin Elmer) plate counter.

### 3. Data Analysis

Data derived from liquid scintillation counting were analyzed by iterative non-linear regression analysis with the use of the Statistical Analysis System (SAS): a program similar to “LIGAND” as described by Munson and Rodbard (Anal. Biochem. 1980, 107, 220-239). Fitting was performed according to formulae described by Feldman (Anal. Biochem. 1972, 48, 317-338). IC₅₀, nH and Kᵢ values were expressed as geometrical mean. For receptors with a low affinity for the test compound, where the highest tested compound concentration inhibited less than 30% of specific radioligand binding, Kᵢ values were determined according to the equation of Cheng and Prusoff (Biochem. Pharmacol. 1973, 22, 2099-2108) and expressed as greater than (>).

The results of the receptor binding studies are expressed as receptor binding constants Kᵢ (5-HT₆) and Kᵢ (5-HT_{2A}), respectively, as described herein before, and given in Table 1.

In these tests, the compounds according to the invention exhibit very good affinities for the 5-HT₆ receptor (Kᵢ<50 nM or <20 nM and frequently <1 nM). Furthermore those compounds bind selectively to the 5-HT₆ receptor, as compared to the affinity at ancillary targets. Representative is Example 1, which was tested against 78 targets (CEREP) other than the 5-HT₆ receptor. Example 1 had a Kᵢ>1 μM at all but the 5-HT_{2B} receptor (71 nM, 173.2 fold selective) and the 5-HT_{2A} receptor (195 nM, 475.6 fold selective). Unlike GSK-21503, compounds of the invention exhibit little affinity for 5-HT_{2A} receptors (Kᵢ>100 nM) as compared to 5-HT₆ receptors.

Table 1 demonstrates that compounds of the invention have an unexpectedly high affinity for 5-HT₆ receptors as compared to 5-HT_{2A} receptors. For example, the compound of Example (1), representative of compounds of the invention, has a 475 fold selectivity for 5-HT₆ over 5-HT_{2A}; while the control, GSK-215083, shows only a 1.16 fold selectivity for 5-HT₆ over 5-HT_{2A}.

TABLE 1

| | Rat | Mk striatum | Human | Human | Selectivity for | Rat |
|---|---|---|---|---|---|---|
| | 5-HT₆ | 5-HT₆ | 5-HT₆ | 5-HT_{2A} | 5-HT₆ v. | microsome |
| Compound | Ki (nM) | Ki (nM) | Ki (nM) | Ki (nM) | 5-HT_{2A} | T½ (mm) |
| 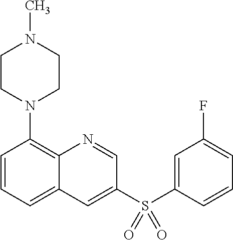 [SMILES: CN1CCN(c2cccc3cc(S(=O)(=O)c4cccc(F)c4)cnc23)CC1] GSK-215083 | 0.16 | 3.2 | 0.34 | 0.395 | 1.16 × | 62.4 |
| 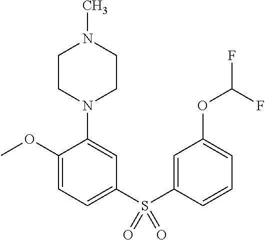 [SMILES: COc1ccc(S(=O)(=O)c2cccc(OC(F)F)c2)cc1N1CCN(C)CC1] Example 1 | 1.50 | 5.0 | 0.41 | 195 | 475.6 × | 10 |

Tables 2 and 3 show that compounds of the invention penetrate the blood/brain barrier and have preferential distribution in the 5-HT₆ receptor-rich regions of the striatum, hippocampus, and frontal cortex. Thus, radiolabeled compounds of the invention are useful as selective 5-HT₆ receptor PET ligands.

TABLE 2

| | Rat PK (0.05 mg/kg, iv) | | | |
|---|---|---|---|---|
| | Min. | Plasma | Free | Free |
| | after | Conc. | [brain] | B/P |
| Compound | dose | (ng/mL) | (ng/g) | Ratio |
| 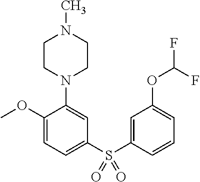 [SMILES: COc1ccc(S(=O)(=O)c2cccc(OC(F)F)c2)cc1N1CCN(C)CC1] Example 1 | 3 5 | 49.2 29.0 | 53.4 55.3 | 1.1 1.7 |

Free B/P = Ratio of free brain concentration divided by free plasma concentration

TABLE 3

| | Min. | | | |
|---|---|---|---|---|
| | after | Striatum/ | Hippo./ | F.Cortex/ |
| [³H]Compound | dose | Cer. | Cer. | Cer. |
| 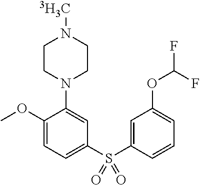 Example 3 | 3 40 | 10.4 2.0 | 3.7 1.4 | 2.8 1.2 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, formulations, or methods of use of the invention, may be made without departing from the spirit and scope thereof.

What is claimed is:

1. A compound of formula (I), 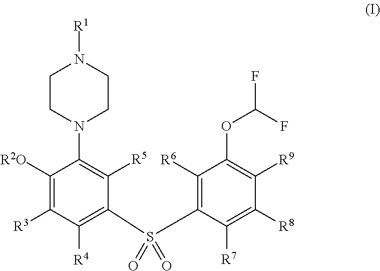
or a pharmaceutically acceptable salt, ester, amide, prodrug, or radiolabeled form thereof, wherein
R¹ and R² are each independently selected from the group consisting of hydrogen, C₁-C₆ alkyl, C₁-C₆ alkenyl, and C₃-C₈ cycloalkyl; and
R³, R⁴, R⁵, R⁶, R⁷, R⁸, and R⁹ are each independently selected from the group consisting of hydrogen, halogen, C₁-C₆ alkyl, C₁-C₆ alkenyl, and C₃-C₈ cycloalkyl.

2. The compound of claim 1, wherein said compound comprises a radiolabel.

3. The compound of claim 1, wherein R¹ comprises a radiolabel.

4. The compound of claim 1, wherein R¹ is hydrogen.

5. The compound of claim 1, wherein R¹ is C₁-C₆ alkyl.

6. The compound of claim 5, wherein R¹ is ¹¹CH₃.

7. The compound of claim 5, wherein R¹ is ¹¹CH₂CH₃.

8. The compound of claim 5, wherein R¹ is ¹¹CH₂CH₂CH₃.

9. The compound of claim 5, wherein R¹ is CH₂CH₂¹⁸F.

10. The compound of claim 5, wherein R¹ is CH₂CH₂CH₂¹⁸F.

11. The compound of claim 5, wherein R¹ is C₁-C₆ hydroxyalkyl.

12. The compound of claim 5, wherein R¹ is —CO₂tBu.

13. The compound of claim 1, wherein R² is hydrogen.

14. The compound of claim 1, where R² comprises a radiolabel.

15. The compound of claim 1, wherein R² is C₁-C₆ alkyl.

16. The compound of claim 15, wherein R² is ¹¹CH₃.

17. The compound of claim 15, wherein R² is ¹¹CH₂CH₃.

18. The compound of claim 15, wherein R² is ¹¹CH₂CH₂CH₃.

19. The compound of claim 15, wherein R² is CH₂CH₂¹⁸F.

20. The compound of claim 15, wherein R² is CH₂CH₂CH₂¹⁸F.

21. The compound of claim 1, wherein R⁷ is ¹⁸F.

22. The compound of claim 1, wherein R⁹ is ¹⁸F.

23. The compound of claim 1, selected from the group consisting of:
1-(5-(3-(difluoromethoxy)phenylsulfonyl)-2-methoxyphenyl)-4-methylpiperazine;
1-(5-(3-(difluoromethoxy)phenylsulfonyl)-2-methoxyphenyl)-4-ethylpiperazine;
1-(5-(3-(difluoromethoxy)phenylsulfonyl)-2-methoxyphenyl)-4-propylpiperazine;
1-(5-(3-(difluoromethoxy)phenylsulfonyl)-2-ethoxyphenyl)-4-methylpiperazine;
1-(5-(3-(difluoromethoxy)phenylsulfonyl)-2-propoxyphenyl)-4-methylpiperazine;
1-(5-(3-(difluoromethoxy)phenylsulfonyl)-2-methoxyphenyl)-4-(2-fluoroethyl)piperazine;
1-(5-(3-(difluoromethoxy)phenylsulfonyl)-2-methoxyphenyl)-4-(3-fluoropropyl)piperazine;
1-(5-(3-(difluoromethoxy)phenylsulfonyl)-2-(2-fluoroethoxy)phenyl)-4-methylpiperazine;
1-(5-(3-(difluoromethoxy)phenylsulfonyl)-2-(3-fluoropropoxy)phenyl)-4-methylpiperazine;
1-(5-(5-(difluoromethoxy)-2-fluorophenylsulfonyl)-2-methoxyphenyl)-4-methylpiperazine;
1-(5-(3-(difluoromethoxy)-4-fluorophenylsulfonyl)-2-methoxyphenyl)-4-methylpiperazine;
1-(5-(3-(difluoromethoxy)-4-fluorophenylsulfonyl)-2-methoxyphenyl)piperazine;
4-(3-(difluoromethoxy)-4-fluorophenylsulfonyl)-2-(4-methylpiperazin-1-yl)phenol;
1-(5-(3-(difluoromethoxy)phenylsulfonyl)-2-methoxyphenyl)-4-[¹¹C]methylpiperazine;
1-(5-(3-(difluoromethoxy)phenylsulfonyl)-2-methoxyphenyl)-4-[1-¹¹C]ethylpiperazine;
1-(5-(3-(difluoromethoxy)phenylsulfonyl)-2-methoxyphenyl)-4-[1-¹¹C]propylpiperazine;
1-(5-(3-(difluoromethoxy)phenylsulfonyl)-2-[¹¹C]methoxyphenyl)-4-methylpiperazine;
1-(5-(3-(difluoromethoxy)phenylsulfonyl)-2-[1-¹¹C]ethoxyphenyl)-4-methylpiperazine;
1-(5-(3-(difluoromethoxy)phenylsulfonyl)-2-[1-¹¹C]propoxyphenyl)-4-methylpiperazine;
1-(5-(3-(difluoromethoxy)phenylsulfonyl)-2-methoxyphenyl)-4-(2-[¹⁸F]fluoroethyl)piperazine;
1-(5-(3-(difluoromethoxy)phenylsulfonyl)-2-methoxyphenyl)-4-(3-[¹⁸F]fluoropropyl)piperazine;
1-(5-(3-(difluoromethoxy)phenylsulfonyl)-2-(2-[¹⁸F]fluoroethoxy)phenyl)-4-methylpiperazine;
1-(5-(3-(difluoromethoxy)phenylsulfonyl)-2-(3-[¹⁸F]fluoropropoxy)phenyl)-4-methylpiperazine;
1-(5-(5-(difluoromethoxy)-2-[¹⁸F]fluorophenylsulfonyl)-2-methoxyphenyl)-4-methylpiperazine;
1-(5-(3-(difluoromethoxy)-4-[¹⁸F]fluorophenylsulfonyl)-2-methoxyphenyl)-4-methylpiperazine; and
1-(5-(3-(difluoromethoxy)phenylsulfonyl)-2-methoxyphenyl)-4-([³H]methyl)-piperazine.

24. A pharmaceutical composition comprising a therapeutically effective amount of a compound of formula (I) according to claim 1, or a pharmaceutically acceptable salt, ester, amide, prodrug, or radiolabeled form thereof, in combination with a pharmaceutically acceptable carrier.

25. Use of a compound of claim 1 as a 5-HT₆ positron emission tomography ligand.

26. The use of claim 25, wherein the compound is selected from the group consisting of:
1-(5-(3-(difluoromethoxy)phenylsulfonyl)-2-methoxyphenyl)-4-[¹¹C]methylpiperazine;
1-(5-(3-(difluoromethoxy)phenylsulfonyl)-2-methoxyphenyl)-4-[1-¹¹C]ethylpiperazine;
1-(5-(3-(difluoromethoxy)phenylsulfonyl)-2-methoxyphenyl)-4-[1-¹¹C]propylpiperazine;
1-(5-(3-(difluoromethoxy)phenylsulfonyl)-2-[¹¹C]methoxyphenyl)-4-methylpiperazine;
1-(5-(3-(difluoromethoxy)phenylsulfonyl)-2-[1-¹¹C]ethoxyphenyl)-4-methylpiperazine;
1-(5-(3-(difluoromethoxy)phenylsulfonyl)-2-[1-¹¹C]propoxyphenyl)-4-methylpiperazine;
1-(5-(3-(difluoromethoxy)phenylsulfonyl)-2-methoxyphenyl)-4-(2-[¹⁸F]fluoroethyl)piperazine;
1-(5-(3-(difluoromethoxy)phenylsulfonyl)-2-methoxyphenyl)-4-(3-[¹⁸F]fluoropropyl)piperazine;
1-(5-(3-(difluoromethoxy)phenylsulfonyl)-2-(2-[¹⁸F]fluoroethoxy)phenyl)-4-methylpiperazine;
1-(5-(3-(difluoromethoxy)phenylsulfonyl)-2-(3-[¹⁸F]fluoropropoxy)phenyl)-4-methylpiperazine;
1-(5-(5-(difluoromethoxy)-2-[¹⁸F]fluorophenylsulfonyl)-2-methoxyphenyl)-4-methylpiperazine;
1-(5-(3-(difluoromethoxy)-4-[¹⁸F]fluorophenylsulfonyl)-2-methoxyphenyl)-4-methylpiperazine; and
1-(5-(3-(difluoromethoxy)phenylsulfonyl)-2-methoxyphenyl)-4-([³H]methyl)-piperazine.

27. The use of claim 26, wherein the compound is selected from the group consisting of:
1-(5-(3-(difluoromethoxy)phenylsulfonyl)-2-methoxyphenyl)-4-[¹¹C]methylpiperazine; and
1-(5-(3-(difluoromethoxy)phenylsulfonyl)-2-[¹¹C]methoxyphenyl)-4-methylpiperazine.

28. A method for imaging 5-HT₆ receptors in a mammal comprising administering to a subject an effective amount of a compound of claim 1, or a pharmaceutically acceptable salt, ester, amide, prodrug, or radiolabeled form thereof.

29. A method of treating or preventing a condition or disorder selected from the group consisting of deficits in memory and cognition and learning, Alzheimer's disease, age-related cognitive decline, mild cognitive impairment, attention deficit/hyperactivity syndrome, schizophrenia, cognitive deficits of schizophrenia, depression, anxiety, obsessive compulsive disorders, Parkinson's disease, epilepsy, migraine, sleep disorders, anorexia, bulimia, irritable bowel syndrome, stroke, spinal or head trauma and head injuries, drug addition, and obesity, comprising administering to a subject in need thereof a therapeutically effective amount of a compound of formula (I) according to claim 1, or a pharmaceutically acceptable salt, ester, amide, prodrug, or radiolabeled form thereof.
